SEQUENCE LISTING (174 sequences)

SEQ ID NO: 1 - DNA, 24 bp, Artificial Sequence
  primer
gagaacaatgagaaccttcaggag

SEQ ID NO: 2 - DNA, 23 bp, Artificial Sequence
  primer
ttctggcgccggttacagaacca

SEQ ID NO: 3 - DNA, 25 bp, Artificial Sequence
  primer
accacttccctcatctctcctccaa

SEQ ID NO: 4 - DNA, 25 bp, Artificial Sequence
  primer
agggtgagggaggcagagacagaaa

SEQ ID NO: 5 - DNA, 22 bp, Artificial Sequence
  primer′
tgcaggaccaaggagactatgt

SEQ ID NO: 6 - DNA, 23 bp, Artificial Sequence
  primer
taggatgatgacaagaagtagcc

SEQ ID NO: 7 - DNA, 27 bp, Artificial Sequence
  primer
atcccatttgcaaagcttctggctggc

SEQ ID NO: 8 - DNA, 27 bp, Artificial Sequence
  primer
tgtgaagcgtctcacaggtccaggatg

SEQ ID NO: 9 - DNA, 25 bp, Artificial Sequence
  primer
aggtcagcagcatcgtggtcaacat

SEQ ID NO: 10 - DNA, 25 bp, Artificial Sequence
  primer
gtggggttgtctttgaataccgcag

SEQ ID NO: 11 - DNA, 22 bp, Artificial Sequence
  primer
ctctgcatcctcaccatcacag

SEQ ID NO: 12 - DNA, 22 bp, Artificial Sequence
  primer
tagccgtagatgtgcagcgtgt

SEQ ID NO: 13 - DNA, 23 bp, Artificial Sequence
  primer
taaacacctgcccatctactcgg

SEQ ID NO: 14 - DNA, 23 bp, Artificial Sequence
  primer
atctcatcatcctgggctgctgg

SEQ ID NO: 15 - DNA, 22 bp, Artificial Sequence
  primer
cggctggtggagtggatcatag

SEQ ID NO: 16 - DNA, 22 bp, Artificial Sequence
  primer
ccctctgttgctgcccatctga

SEQ ID NO: 17 - DNA, 18 bp, Artificial Sequence
  primer
gccttaccacatccgctc

SEQ ID NO: 18 - DNA, 22 bp, Artificial Sequence
  primer
tcacactcttccgtcacattgc

SEQ ID NO: 19 - DNA, 22 bp, Artificial Sequence
  primer
agacatcgcactgactgagaac

SEQ ID NO: 20 - DNA, 22 bp, Artificial Sequence
  primer
gacgggtcactatctgtgcaac

SEQ ID NO: 21 - DNA, 20 bp, Artificial Sequence
  primer
cttcaagccagaggcctacg

SEQ ID NO: 22 - DNA, 20 bp, Artificial Sequence
  primer
ccgcctctgtcttcttcagc

SEQ ID NO: 23 - DNA, 24 bp, Artificial Sequence
  primer
tgcagccaaagtgaagagggaaga

SEQ ID NO: 24 - DNA, 24 bp, Artificial Sequence
  primer
catagcgagcagcccaaagaagaa

SEQ ID NO: 25 - DNA, 24 bp, Artificial Sequence
  primer
tgcttgaatgtgctgatgacaggg

SEQ ID NO: 26 - DNA, 25 bp, Artificial Sequence
  primer
aaggcaagtcagcaggcatctcatc

SEQ ID NO: 27 - DNA, 25 bp, Artificial Sequence
  primer
gtactggtctcagcagattgaggag

SEQ ID NO: 28 - DNA, 25 bp, Artificial Sequence
  primer
gcttctgctggcttaatgcctcaga

SEQ ID NO: 29 - DNA, 20 bp, Artificial Sequence
  primer
atggccgagcagaaccggaa

SEQ ID NO: 30 - DNA, 20 bp, Artificial Sequence
  primer
ccatgagccgctggtactcc

SEQ ID NO: 31 - DNA, 20 bp, Artificial Sequence
  primer
tcatcgctcaggaggtcctt

SEQ ID NO: 32 - DNA, 21 bp, Artificial Sequence
  primer
ctgttgccagagatggaggtt

SEQ ID NO: 33 - DNA, 20 bp, Artificial Sequence
  primer
gaagactcgcatccgaatgg

SEQ ID NO: 34 - DNA, 21 bp, Artificial Sequence
  primer
cgcaggataggaggaagagac

SEQ ID NO: 35 - DNA, 20 bp, Artificial Sequence
  primer
ttagctgaattcgcgtgtgg

SEQ ID NO: 36 - DNA, 22 bp, Artificial Sequence
  primer
gaggaagtgaatgagccggtta

SEQ ID NO: 37 - DNA, 22 bp, Artificial Sequence
  primer
gcgccatatccaacagtgacag

SEQ ID NO: 38 - DNA, 23 bp, Artificial Sequence
  primer
gccagcagttgcattgacatata

SEQ ID NO: 39 - DNA, 22 bp, Artificial Sequence
  primer
gtaaaagcaaagacgggactgg

SEQ ID NO: 40 - DNA, 22 bp, Artificial Sequence
  primer
atgatggatgttgcctaatgag

SEQ ID NO: 41 - DNA, 19 bp, Artificial Sequence
  primer
gctggaaggcggatgtttc

SEQ ID NO: 42 - DNA, 20 bp, Artificial Sequence
  primer
tcgttttgggattaatgccc

SEQ ID NO: 43 - DNA, 22 bp, Artificial Sequence
  primer
tggtggcatctgacactagtga

SEQ ID NO: 44 - DNA, 24 bp, Artificial Sequence
  primer
cttccagtataaggctccaaaagc

SEQ ID NO: 45 - DNA, 22 bp, Artificial Sequence
  primer
aggaagcagtctgtgtagtgtg

SEQ ID NO: 46 - DNA, 22 bp, Artificial Sequence
  primer
gatggtagtagagggatgtggg

SEQ ID NO: 47 - DNA, 23 bp, Artificial Sequence
  primer
cttgggcaggctgatagttttta

SEQ ID NO: 48 - DNA, 23 bp, Artificial Sequence
  primer
tttgtacttggctcattgctcct

SEQ ID NO: 49 - DNA, 23 bp, Artificial Sequence
  primer
tagttaatctcctcagacagtaa

SEQ ID NO: 50 - DNA, 23 bp, Artificial Sequence
  primer
gctactaacctacctattcattt

SEQ ID NO: 51 - DNA, 18 bp, Artificial Sequence
  primer
agaggggaattcctggag

SEQ ID NO: 52 - DNA, 20 bp, Artificial Sequence
  primer
ctgaggaccaggactctcta

SEQ ID NO: 53 - DNA, 20 bp, Artificial Sequence
  primer
aacgccacacagtgccaaat

SEQ ID NO: 54 - DNA, 20 bp, Artificial Sequence
  primer
gcatgggtccttgtaaagct

SEQ ID NO: 55 - DNA, 21 bp, Artificial Sequence
  primer
ataagcttgatctcgtcttcc

SEQ ID NO: 56 - DNA, 21 bp, Artificial Sequence
  primer
cttgctaggatgtaacaaagc

SEQ ID NO: 57 - DNA, 20 bp, Artificial Sequence
  primer
gacagactgcaagaggctcc

SEQ ID NO: 58 - DNA, 20 bp, Artificial Sequence
  primer
ggagaggcgaggaagtcacc

SEQ ID NO: 59 - DNA, 18 bp, Artificial Sequence
  primer
aagttccgcaaggtgcag

SEQ ID NO: 60 - DNA, 19 bp, Artificial Sequence
  primer
ttggcaagcagtgaggttc

SEQ ID NO: 61 - DNA, 18 bp, Artificial Sequence
  primer
ccttccgcatgtttgacc

SEQ ID NO: 62 - DNA, 20 bp, Artificial Sequence
  primer
gcccctcattcctctttctc

SEQ ID NO: 63 - DNA, 20 bp, Artificial Sequence
  primer
caaagatctgctcctcgctc

SEQ ID NO: 64 - DNA, 19 bp, Artificial Sequence
  primer
agtggtggctcccacctag

SEQ ID NO: 65 - DNA, 20 bp, Artificial Sequence
  primer
aagccaatttttctgcactg

SEQ ID NO: 66 - DNA, 21 bp, Artificial Sequence
  primer
aacaatgttttctgcacaagc

SEQ ID NO: 67 - DNA, 24 bp, Artificial Sequence
  primer
gccttttgatactcttactgtggc

SEQ ID NO: 68 - DNA, 22 bp, Artificial Sequence
  primer
caggagaaagattgggaagtgg

SEQ ID NO: 69 - DNA, 20 bp, Artificial Sequence
  primer
ccaagtcattgttggataag

SEQ ID NO: 70 - DNA, 21 bp, Artificial Sequence
  primer
cttagatgagttttctttcac

SEQ ID NO: 71 - DNA, 22 bp, Artificial Sequence
  primer
ccagtctagttcctgatgaccc

SEQ ID NO: 72 - DNA, 18 bp, Artificial Sequence
  primer
caaggccgttcattgtcc

SEQ ID NO: 73 - DNA, 17 bp, Artificial Sequence
  primer
agtcagctcagcagcag

SEQ ID NO: 74 - DNA, 16 bp, Artificial Sequence
  primer
ggggtcaacaccacag

SEQ ID NO: 75 - DNA, 21 bp, Artificial Sequence
  primer
atcagaactgcaatctgcaca

SEQ ID NO: 76 - DNA, 22 bp, Artificial Sequence
  primer
agaagatccctgtcacaattcc

SEQ ID NO: 77 - DNA, 19 bp, Artificial Sequence
  primer
cgcctgtagtcccagctac

SEQ ID NO: 78 - DNA, 20 bp, Artificial Sequence
  primer
gatcttggctcactgcaagc

SEQ ID NO: 79 - DNA, 22 bp, Artificial Sequence
  primer
gcactcttccagccttccttcc

SEQ ID NO: 80 - DNA, 23 bp, Artificial Sequence
  primer
tcaccttcaccgttccagttttt

SEQ ID NO: 81 - DNA, 23 bp, Artificial Sequence
  primer
ctaggcatcacctgtgccatacc

SEQ ID NO: 82 - DNA, 25 bp, Artificial Sequence
  primer
cagtgaccagttcatcagattcatc

SEQ ID NO: 83 - DNA, 22 bp, Artificial Sequence
  primer
ccaggaccaaagggacagaaag

SEQ ID NO: 84 - DNA, 22 bp, Artificial Sequence
  primer
ttcaccaggttcaccaggattg

SEQ ID NO: 85 - DNA, 21 bp, Artificial Sequence
  primer
gctgttatgggtgaaactctg

SEQ ID NO: 86 - DNA, 21 bp, Artificial Sequence
  primer
ataaggtggagatgcaggctc

SEQ ID NO: 87 - DNA, 21 bp, Artificial Sequence
  primer
gcctttgtgaaccaacacctg

SEQ ID NO: 88 - DNA, 21 bp, Artificial Sequence
  primer
gttgcagtagttctccagctg

SEQ ID NO: 89 - DNA, 19 bp, Artificial Sequence
  primer
cccatggatgaagtctacc

SEQ ID NO: 90 - DNA, 19 bp, Artificial Sequence
  primer
gtcctcctcctttttccac

SEQ ID NO: 91 - DNA, 26 bp, Artificial Sequence
  primer
ctcgagggtagaaaggatgacgcctc

SEQ ID NO: 92 - DNA, 27 bp, Artificial Sequence
  primer
acgcgtgaatgggattatggggtggtg

SEQ ID NO: 93 - DNA, 20 bp, Artificial Sequence
  primer
atggtgcagctgagtcctcc

SEQ ID NO: 94 - DNA, 20 bp, Artificial Sequence
  primer
tctcattcttgctgagcttc

SEQ ID NO: 95 - DNA, 20 bp, Artificial Sequence
  primer
agccggcacctgttgtgcaa

SEQ ID NO: 96 - DNA, 20 bp, Artificial Sequence
  primer
tgacttctcctgcatgcact

SEQ ID NO: 97 - DNA, 20 bp, Artificial Sequence
  primer
atgcacggcatctgggaatc

SEQ ID NO: 98 - DNA, 24 bp, Artificial Sequence
  primer
gctactgtcctgcaagttgctgtc

SEQ ID NO: 99 - DNA, 21 bp, Artificial Sequence
  primer
accccactgtggcggcgagat

SEQ ID NO: 100 - DNA, 16 bp, Artificial Sequence
  primer
cacagcactagagacc

SEQ ID NO: 101 - DNA, 20 bp, Artificial Sequence
  primer
cagtctgaccagcgtgaaaa

SEQ ID NO: 102 - DNA, 20 bp, Artificial Sequence
  primer
ggccatccaaatctgtccta

SEQ ID NO: 103 - DNA, 21 bp, Artificial Sequence
  primer
gctggttcaagggctttattc

SEQ ID NO: 104 - DNA, 21 bp, Artificial Sequence
  primer
tggggcaggtggagctgggcg

SEQ ID NO: 105 - DNA, 20 bp, Artificial Sequence
  primer
aggggtctacatggcaactg

SEQ ID NO: 106 - DNA, 20 bp, Artificial Sequence
  primer
cgaccactttgtcaagctca

SEQ ID NO: 107 - DNA, 23 bp, Artificial Sequence
  primer
ttsccaggcaaagagggctggac

SEQ ID NO: 108 - DNA, 23 bp, Artificial Sequence
  primer
ggctgtgtgagcaagatcctagg

SEQ ID NO: 109 - DNA, 22 bp, Artificial Sequence
  primer
taacagtgcccttttcatctcc

SEQ ID NO: 110 - DNA, 23 bp, Artificial Sequence
  primer
ctgtgccactgagatataggtcc

SEQ ID NO: 111 - DNA, 22 bp, Artificial Sequence
  primer
aaacaaagcaaatgttcagtgg

SEQ ID NO: 112 - DNA, 19 bp, Artificial Sequence
  primer
tgggtccccaaaagcttag

SEQ ID NO: 113 - DNA, 20 bp, Artificial Sequence
  primer
tcagcaggcaatagattggg

SEQ ID NO: 114 - DNA, 20 bp, Artificial Sequence
  primer
aaaggaaaggccgtctaggg

SEQ ID NO: 115 - DNA, 23 bp, Artificial Sequence
  primer
gatctacctagtttaatagaccc

SEQ ID NO: 116 - DNA, 22 bp, Artificial Sequence
  primer
tgtactagttgagaaagctgat

SEQ ID NO: 117 - DNA, 18 bp, Artificial Sequence
  primer
agtgagcaaaactgccgc

SEQ ID NO: 118 - DNA, 20 bp, Artificial Sequence
  primer
gaagatgctgctgtgcttcc

SEQ ID NO: 119 - DNA, 22 bp, Artificial Sequence
  primer
cttcacaacatcacctgctagc

SEQ ID NO: 120 - DNA, 21 bp, Artificial Sequence
  primer
acaggttggggtacttcatcc

SEQ ID NO: 121 - DNA, 20 bp, Artificial Sequence
  primer
tgaaatcctgggtctcttgg

SEQ ID NO: 122 - DNA, 20 bp, Artificial Sequence
  primer
gcaatgcaagagcaaacaaa

SEQ ID NO: 123 - DNA, 20 bp, Artificial Sequence
  primer
gactttccagcagtcccata

SEQ ID NO: 124 - DNA, 20 bp, Artificial Sequence
  primer
gtttacttcctgcagggaac

SEQ ID NO: 125 - DNA, 20 bp, Artificial Sequence
  primer
ctacaggggcacttaaccca

SEQ ID NO: 126 - DNA, 20 bp, Artificial Sequence
  primer
agagctgaatcgctcagagc

SEQ ID NO: 127 - DNA, 21 bp, Artificial Sequence
  primer
actctggaaggttctcatgtg

SEQ ID NO: 128 - DNA, 21 bp, Artificial Sequence
  primer
aggtgtctccatctctgtctc

SEQ ID NO: 129 - DNA, 20 bp, Artificial Sequence
  primer
cttttcgcgcgcccagcatt

SEQ ID NO: 130 - DNA, 19 bp, Artificial Sequence
  primer
gatcttcctgtccctcgag

SEQ ID NO: 131 - DNA, 20 bp, Artificial Sequence
  primer
agaacagcacgtacacagcc

SEQ ID NO: 132 - DNA, 20 bp, Artificial Sequence
  primer
cctccgaagaaacagcaaga

SEQ ID NO: 133 - DNA, 21 bp, Artificial Sequence
  primer
agaaggtgaccagcctaacgg

SEQ ID NO: 134 - DNA, 21 bp, Artificial Sequence
  primer
tctgagcactgtgacgttctg

SEQ ID NO: 135 - DNA, 19 bp, Artificial Sequence
  primer
gctctggacaaaccctgca

SEQ ID NO: 136 - DNA, 20 bp, Artificial Sequence
  primer
agtgggagtccaagactcag

SEQ ID NO: 137 - DNA, 21 bp, Artificial Sequence
  primer
atttattttgtccttctccca

SEQ ID NO: 138 - DNA, 23 bp, Artificial Sequence
  primer
gttaacaacttttgtgtgccaac

SEQ ID NO: 139 - DNA, 21 bp, Artificial Sequence
  primer
tgaacctgtttgcatccttca

SEQ ID NO: 140 - DNA, 21 bp, Artificial Sequence
  primer
acttggcaagcctgcatttga

SEQ ID NO: 141 - DNA, 23 bp, Artificial Sequence
  primer
tcagtttatcctgcccactgatt

SEQ ID NO: 142 - DNA, 23 bp, Artificial Sequence
  primer
gggagctgatgaaactcacaaat

SEQ ID NO: 143 - DNA, 23 bp, Artificial Sequence
  primer
acagagctgcctctcggtggcca

SEQ ID NO: 144 - DNA, 25 bp, Artificial Sequence
  primer
ttcccttctgccggagaggctacgc

SEQ ID NO: 145 - DNA, 23 bp, Artificial Sequence
  primer
tagcccagtcattcctaaaccag

SEQ ID NO: 146 - DNA, 23 bp, Artificial Sequence
  primer
ctgtcctaagaggcaagaaacca

SEQ ID NO: 147 - DNA, 22 bp, Artificial Sequence
  primer
aggacttgcactttccagcttg

SEQ ID NO: 148 - DNA, 22 bp, Artificial Sequence
  primer
tcccgtccagtgtcaggttatc

SEQ ID NO: 149 - DNA, 25 bp, Artificial Sequence
  primer
tttttaatgaggtggctttctaaca

SEQ ID NO: 150 - DNA, 19 bp, Artificial Sequence
  primer
agcaatccgaggaaacgag

SEQ ID NO: 151 - DNA, 20 bp, Artificial Sequence
  primer
tcagggaaagaaagtggtgc

SEQ ID NO: 152 - DNA, 20 bp, Artificial Sequence
  primer
aagaaggagccctgatttcc

SEQ ID NO: 153 - DNA, 20 bp, Artificial Sequence
  primer
tgatgtagacgctgctggtc

SEQ ID NO: 154 - DNA, 20 bp, Artificial Sequence
  primer
ggctccagcaccatgatact

SEQ ID NO: 155 - DNA, 20 bp, Artificial Sequence
  primer
ctgctgatccttcccgatac

SEQ ID NO: 156 - DNA, 20 bp, Artificial Sequence
  primer
attggctgtgaacttggacc

SEQ ID NO: 157 - DNA, 20 bp, Artificial Sequence
  primer
ccactgccaatacaacttcc

SEQ ID NO: 158 - DNA, 20 bp, Artificial Sequence
  primer
gcaagagcagcatccacatc

SEQ ID NO: 159 - DNA, 21 bp, Artificial Sequence
  primer
catctacacagtcaacctcta

SEQ ID NO: 160 - DNA, 21 bp, Artificial Sequence
  primer
ctaaagaaacacaagacaaaa

SEQ ID NO: 161 - DNA, 20 bp, Artificial Sequence
  primer
agaccaacaacccaaacagc

SEQ ID NO: 162 - DNA, 20 bp, Artificial Sequence
  primer
gtcaccagagcttctctggg

SEQ ID NO: 163 - DNA, 18 bp, Artificial Sequence
  primer
aatcattgccatcgtgtg

SEQ ID NO: 164 - DNA, 20 bp, Artificial Sequence
  primer
cgcggcttaaaatctggtat

SEQ ID NO: 165 - DNA, 20 bp, Artificial Sequence
  primer
tggacgctgtgcagagcctg

SEQ ID NO: 166 - DNA, 20 bp, Artificial Sequence
  primer
caggtcctgggctttgagcg

SEQ ID NO: 167 - DNA, 21 bp, Artificial Sequence
  primer
aactggaactgacacaccagg

SEQ ID NO: 168 - DNA, 19 bp, Artificial Sequence
  primer
cctatgcaacccccagtcc

SEQ ID NO: 169 - DNA, 20 bp, Artificial Sequence
  primer
cagttctgctcctctccagg

SEQ ID NO: 170 - DNA, 19 bp, Artificial Sequence
  primer
ccatcctcctgacacctcc

SEQ ID NO: 171 - DNA, 23 bp, Artificial Sequence
  primer
gcatctgcgagttcatcagcaac

SEQ ID NO: 172 - DNA, 22 bp, Artificial Sequence
  primer
gggtccagggtccagtagttgc

SEQ ID NO: 173 - DNA, 25 bp, Artificial Sequence
  primer
cctgctctcttgtaatgatatagcc

SEQ ID NO: 174 - DNA, 25 bp, Artificial Sequence
  primer
gagactagaactgagctgtttgtcc

(19) United States
(12) Patent Application Publication
(10) Pub. No.: US 2010/0047908 A1
(43) Pub. Date: Feb. 25, 2010

(54) MONOCYTE-DERIVED STEM CELLS

(76) Inventors: Glenn E. Winnier, The Woodlands, TX (US); Brian S. Newsom, Spring, TX (US); Donna R. Rill, The Woodlands, TX (US); Jim C. Williams, The Woodlands, TX (US)

Correspondence Address: POLSINELLI SHUGHART PC, 700 W. 47TH STREET, SUITE 1000, KANSAS CITY, MO, 64112-1802, US

(21) Appl. No.: 12/299,588

(22) PCT Filed: May 4, 2007
(86) PCT No.: PCT/US07/68291

§ 371 Date: Nov. 4, 2008

Related U.S. Application Data
(60) Provisional application No. 60/746,609, filed on May 5, 2006.

(51) Int. Cl. C12N 5/074; C12N 5/00

(52) U.S. Cl. 435/372; 435/375; 435/325

(57) ABSTRACT

Methods for generating multipotent stem cells from adult peripheral blood monocytes are provided. Monocytes may be de-differentiated into monocyte-derived stem cells (MDSCs) by contacting the monocyte with the de-differentation factors, leukocyte inhibitory factor, macrophage colony-stimulating factor, or a combination thereof. The MDSCs may be differentiated into many different types of cells upon contact with the appropriate differentiation factors. Also provided are compositions comprising the MDSCs or differentiated cells derived from the MDSCs.

### FIELD OF THE INVENTION

This invention relates to methods of generating adult stem cells and compositions of the resultant stem cells.

### BACKGROUND OF THE INVENTION

Pluripotent or multipotent stem cells are a valuable resource for research, drug discovery and therapeutic treatments, including transplantation (Lovell-Badge, 2001, Nature, 414:88-91; Donovan et al., 2001, Nature, 414:92-97; Griffith et al., 2002, Science, 295:1009-1014; Weissman, 2002, N. Engl. J. Med., 346:1576-1579). These cells, or their mature progeny, can be used to study signaling events that regulate differentiation processes, identify and test drugs for lineage-specific beneficial or cytotoxic effects, or replace tissues damaged by disease or an environmental impact. The current state of stem cell biology and the medicinal outlook, however, are not without drawbacks or free from controversy.

The use of pluripotent or multipotent stem cells from fetuses, umbilical cords or embryonic tissues derived from in vitro fertilized eggs raises ethical and legal questions in the case of human materials, poses a risk of transmitting infections and/or may be ineffective because of immune rejection. In particular, embryonic stem cells have a number of disadvantages. For example, embryonic stem cells may pass through several intermediate stages before becoming the cell type needed to treat a particular disease. In addition, embryonic stem cells may be rejected by the recipient's immune system since it is possible that the immune profile of the specialized cells would differ from that of the recipient.

One way to circumvent these problems is by exploiting autologous stem cells, preferably from an easily accessible tissue such as peripheral blood. The most widely used source of adult stem cells is derived from bone marrow or peripheral blood. The mesenchymal compartment contains several cell populations, including mesenchymal stem cells (MSCs) that are capable of differentiating into a variety of different cell types including adipogenic, osteogenic, chondrogenic, and myogenic cells when cultured under the appropriate growth conditions (Pittenger et al., 1999, Science, 284:143-147). Early studies using bone marrow stromal cells for tissue repair focused on the repair of bone defects (Takagi and Urist, 1982, Clin Orthop, 171:224-231). However, more recent studies have applied bone marrow stem cells to repair a variety of damaged tissue types, including cartilage (Wakitani et al., 2002, Osteoarthritis Cartilage, 10: 199-206), myocardium (Orlic et al., 2003, Pediatr Transplant, 7 Suppl 3:86-88; Terai et al., 2002, J Gastroenterol, 37 Suppl 14:162-163), and most recently diabetes (lanus et al., 2003, J Clin Invest, 2003; 111:843-850). Recent studies have demonstrated that bone marrow contains cells that appear to have the ability to trans-differentiate into mature cells belonging to cell lineages other than those of the blood (Laggase et al., 2000, Nature Med, 6:1229-1234; Orlic et al., 2001, Nature, 410:640-641; Korbling et al., N. Engl J Med, 346:738-746). However, recent studies have suggested that these cells undergo a trans-differentiation that results from the fusion of the stem cell with resident tissue cells (Terada et al., 2002, Nature, 416:542-545; Ying et al., 2002, Nature, 416:545-548). But, autologous bone marrow procurement has potential limitations including low yields, costly processes, and painful procedures. An alternate source of autologous adult stem cells that is obtainable in large quantities, under local anesthesia, with minimal discomfort would be advantageous.

Thus, needs exist in the art to isolate, culture, sustain, propagate, and differentiate adult stem cells, particularly human adult stem cells that are relatively accessible in order to develop cell types suitable for a variety of uses. Such uses may include the use of autologous stem cells for the treatment of diseases and amelioration of symptoms of diseases.

### SUMMARY OF THE INVENTION

Provided herein is a method for generating a stem cell. A monocyte may be contacted with a de-differentiation factor, such as leukocyte inhibitory factor (LIF), macrophage colony-stimulating factor (M-CSF), or a combination thereof, which may cause the monocyte to de-differentiated into a stem cell. The monocyte may be isolated from mammalian peripheral blood. The mammal may be a human. The mammal may be an adult.

The stem cell may be generated after 4-8 days in culture. A plurality of stem cells may be generated. The plurality of cells may comprise more than 1×10⁶ cells. The stem cell may be contacted with a cryopreservative agent and deep-frozen.

The stem cell may express CD117, DPPA5, HES-1, OCT-4, SSEA4, or a combination thereof. The monocyte may not express CD117, DPPA5, Oct-4, SSEA-4, or a combination thereof. The stem cell may have any of the following characteristics: CD4+, CD11b+, CD14+, CD45+, CD90−, CD117+, DPPA5+, HES-1+, Oct-4+, SSEA-4+, CD34−, CD135− or a combination thereof.

### BRIEF DESCRIPTION OF THE DRAWINGS

FIG. 1 depicts graphs illustrating the growth of monocyte-derived stem cells in different medium formulations and different concentration of fetal bovine calf serum (FBS). Panel A presents the percent confluency on day 3 and panel B presents the percent confluency on day 6.

FIG. 2 depicts a graph illustrating the total cell count in three different preparations of monocyte-derived stem cells from days 1 to 15 of culture.

FIG. 3 depicts a graph illustrating the average cell diameter in two different preparations of monocyte-derived stem cells from days 1 to 8 of culture.

FIG. 4 depicts DNA histograms of monocyte-derived stem cells. Panel A presents the DNA profile of large adherent cells (MDSCs) on day 2 of culture, and panel B present the DNA profile of large adherent cells (MDSCs) on day 6 in culture. The percent of cells in each phase of the cell cycle is presented below each histogram.

FIG. 5 depicts graphs illustrating the percentage of cells in each phase of the cell cycle from days 2 to 6. Plotted are the percentage of small non-adherent cells (NA), large NA, small adherent (Ad), and large Ad cells. Panel A presents the percentage of cells in G0/G1 phase. Panel B presents the percentage of cells in S phase. Panel C present the percentage of cells in G2/M phase. Panel D presents the percentage of aneuploid cells.

FIG. 6 depicts photomicrographs of the expression of cell lineage markers in monocyte-derived stem cells on day 6. Cell nuclei were stained with DAPI (blue). Panel A shows low expression of CD14 (green). Panel B shows no expression of CD34. Panel C shows no expression of CD90. Panel D shows no expression of Nestin. Panel E shows high expression of HLA. Panel F shows low expression of osteocalcin.

FIG. 7 depicts photomicrographs of the expression of stem cell markers in monocyte-derived stem cells on day 5. Cell nuclei were stained with DAPI (blue). Panel A shows expression of HES 1 (green). Panel B shows expression of SSEA4. Panel C shows expression of CD117. Panel D shows control cells.

FIG. 8 depicts a graph illustrating the relative expression of specific genes in monocyte-derived stem cells from day 1 to day 15. Gene expression was analyzed by real-time PCR.

FIG. 9 depicts a histograms showing the expression of CD11b (A), CD 135 (B), CD14 (C), and CD 123 (D) (dotted black lines), compared to IgG (solid dark line, A-D) in buffy coat #49 of MDSCs at day 9 compared to IgG as measured using antibody staining and flow cytometry.

FIG. 10 depicts a histograms showing the expression of CD11b (A), CD135 (B), CD14 (C), and CD123 (D) (dotted black lines), compared to IgG (solid dark line, A-D) in buffy coat #66 of MDSCs at day 21 cultured in de-differentiation medium as measured using antibody staining and flow cytometry.

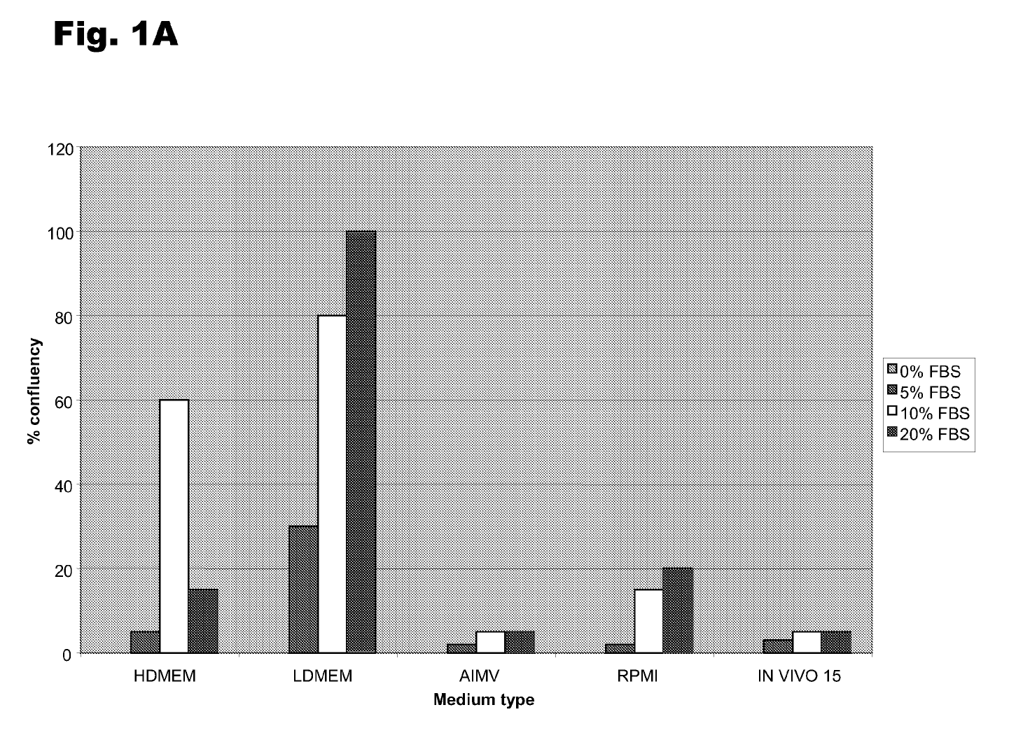

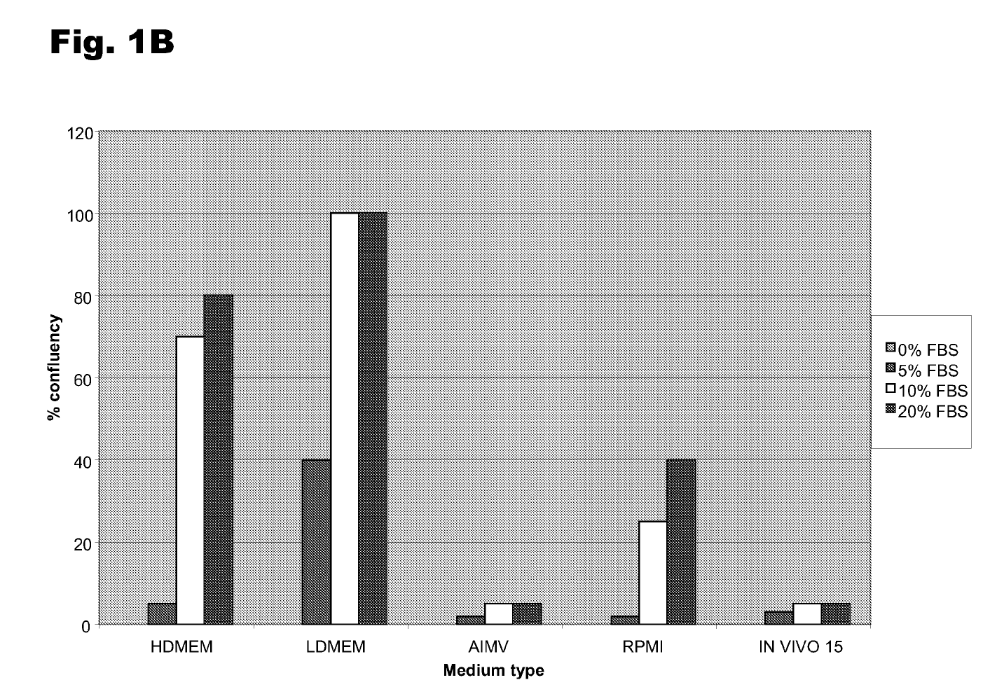

### DETAILED DESCRIPTION OF THE INVENTION

### 1. A Method for Generating a Stem Cell

Provided herein is a method for generating a multipotent stem cell. The stem cell may be generated by contacting a monocyte with a de-differentiation factor. The de-differentiation factor may be leukocyte inhibitory factor (LIF), macrophage colony-stimulating factor (M-CSF), and a combination thereof. Exposure to the de-differentiation factor may cause the monocyte to de-differentiate into a stem cell. The stem cell may express a marker, such as CD117, DPPA5, HES-1, Oct-4, SSEA-4, and combinations thereof.

a. Monocyte

The monocyte may be derived from peripheral blood, which may be from a mammal. The mammal may be a human, a research animal, or a domesticated livestock or pet. The mammal may be an adult.

The monocyte may be derived from peripheral blood using, for example, a single-step discontinuous Ficoll gradient fractionation procedure. The monocyte may be isolated from peripheral blood using another method known to a skilled artisan. The monocyte may be freshly isolated or may be from a frozen preparation.

b. Growth and De-differentiation

The monocyte may be grown in a culture medium. The culture medium may be AIM V (Invitrogen). The monocyte may be seeded on coated or uncoated polystyrene culture plates, dishes, or slides. The culture vessel may be coated with fibronectin, gelatin, collagen, polylysine, or L-ornithine. The cells may be seeded on untreated FALCON integrid vacuum-gas plasma treated plates or dishes. The density of cells to be seeded may range from approximately 1×10⁶/ml to approximately 2×10⁶/ml.

The monocyte may be contacted with leukocyte inhibitory factor (LIF) and macrophage colony-stimulating factor (M-CSF). The concentration of LIF may be from approximately 10 ng/ml to approximately 25 ng/ml. The concentration of M-CSF may range from approximately 5 ng/ml to approximately 50 ng/ml. For example, the concentrations of LIF and M-CSF may be 10 ng/ml and 25 ng/ml, respectively. The de-differentiation factors, LIF and M-CSF, may be provided to the cells in the presence of a culture medium.

The culture medium may be LDMEM (low glucose DMEM), HDMEM (high glucose DMEM), DMEM/F12, or Megacell DMEM/F12. The culture medium may be supplemented with 10-20% fetal bovine calf serum (FBS). Cultures may also be supplemented with 10-20% human AB serum.

The cultures may also be grown in serum free conditions. The growth and de-differentiation of cells may be conducted using Megacell DMEM/F12 medium without FBS (fetal bovine serum). The medium may be supplemented with sodium selenite, rh-Insulin, human transferrin, fatty acids, 4,500 mg/L D-glucose, 4 mM L-glutamine, penicillin-streptomyocin, and combinations thereof. Other media in similar serum-free conditions may be utilized

M-CSF and LIF may be natural or synthetic, and may be used in a purified or unpurified state. Further, the M-CSF or LIF may be a holoprotein or may be active subunits or fragments that exhibit a mitogenic effect on isolated monocytes. Conventional titration assays may be used to determine the effective concentration of M-CSF or LIF.

The monocyte may be cultured under growth conditions well known in the art to propagate the cells, such as 37° C., 5% CO₂. The culture medium containing LIF and M-CSF may be changed every three days. Cell growth and de-differentiation parameters may be analyzed by dispersing and collecting the cells. The cells may be dispersed by addition of approximately 0.5% lidocaine with gentle scraping. The cells may also be dispersed by addition of trypsin/EDTA or collagenase with gentle scraping. The dispersed cells may be counted using a cell counter, examined under a microscope, stained for cell markers, or used for molecular analyses.

c. Monitoring De-Differentiation

The de-differentiation of a monocyte into a stem cell may be monitored by a variety of methods well known in the art. Changes in a parameter between an untreated control cell and a LIF/M-CSF-treated cell may be an indication that the cell has de-differentiated. Changes in the rate of proliferation may indicate de-differentiation. A control monocyte may be essentially quiescent, whereas a de-differentiated cell may have an increase in the rate of cell proliferation. Changes in the rate of proliferation may be measured by counting the total number of cells in the two populations. Changes in the cell cycle may also indicate that the cells have undergone a de-differentiation process. A control monocyte may be in the GO/GI phase of the cell cycle, whereas a cell undergoing de-differentiation may be in the S or G2/M phases of the cell cycle. Changes in the cell cycle may be monitored by flow cytometry. Changes in the cell cycle also may be monitored by the incorporation of BrdU into newly synthesized DNA or by staining for a cell proliferation antigen, such as PCNA or cyclins.

Changes in the expression of a specific marker may also indicate de-differentiation. Expression of specific markers may be monitored at the level of protein by staining with antibodies against the marker. Cell surface or intracellular markers that may be examined include, but are not limited to, CD3, CD11b (MAC-1), CD14, CD31, CD34, CD45, CD90 (Thy-1), CD117 (c-kit receptor), CD123 (IL3R), CD133, 135 (Flk-2), DPPA4, HES-1, HLAabc, MAP-2, nestin, Oct-4, osteocalcin, pankeratin, SSEA4, VEGF-R3, VEGFR (KDR), and vWF. The cells may be fixed and immunostained using procedures well known in the art. For example, a primary antibody may be labeled with a fluorophore or chromophore for direct detection, or a primary antibody may be detected with a secondary antibody that is labeled with a fluorophore or chromophore. The fluorophore may be fluorescein, FITC, rhodamine, Texas Red, Cy-3, Cy-5, Cy-5.5, Alexa⁴⁸⁸, Alexa⁵⁹⁴, QuantumDot⁵²⁵, QuantumDot⁵⁶⁵, or QuantumDot⁶⁵⁵. The fluorescently labeled cell may be examined under a fluorescent light microscope, a confocal microscope, or a multi-photon microscope. The labeled cell may also be analyzed by flow cytometry

RT-PCR and quantitative PCR methods may be used to monitor the changes in gene expression. RNA may be isolated from the cells using procedures known to one skilled in the art. Similarly, PCR may be performed using conditions and parameters well known in the art. Gene transcripts that may be amplified during PCR include ABCG2, AC133, ACTB, AFP, ALB, ANF, ATP2A2, BMP-4, BNP, carboxypeptidase, CD4, CD9, CD10, CD11B, CD13, CD14, CD31, CD33, CD34, CD38, CD45, CD90 (THY1), CD105, CD117 (c-kit receptor), CD123 (IL3R), CD133, CD135 (Flk-2), CDX-2, CK18, CK19, col2a1, CXCR3, CXCR4, DPPA5, E-cadherin, Flk-1, GAD, GAPDH, GATA-2, GATA-3, GATA4, GENESIS, GFAP, GLP-1R, glucagon, Glut2, HLA-A, HNF-3B, IAPP, IGF2, insulin, IPF1, GLP-1, Islet1, keratin, MAP2, MBP, myosin heavy chain, nestin, neurogenin, NGN3, NKX-2.2, NKX2.5, NSE, Oct4, osteocalcin, osteopontin, pancreatic amylase, PAX-4, PAX6, PDGFRB, PDX-1, PPAR2, REX-1, SCF, SM1, SM22A, somatostatin, SOX-2, TAL-1, TAU, TBX-5, TIE-2, troponin, VE-cadherin, and VEGFR2 (KDR). Changes in gene expression (increases or decreases) between two cells exposed to different conditions may indicate that the state of differentiation has changed between the two cells.

The expression of some cell markers may not change during differentiation. Markers whose expression may be detected in both monocytes and stem cells include, AC133, ANF, BMP-4, BNP, CD4, CD9, CD10, CD11b, CD14, CD31, CD33, CD45, CD71, CD90, CD123 (IL3R), CD133, CD135 (Flk-2), CK18, CK19, C-peptide, CXCR3, GATA4, GLUT2, HLAabc, IAPP, Islet-1, osteopontin, and PDX-1. However, the expression of some of these markers may increase or decrease during the cells differentiation. Markers whose expression may not be detected in both monocytes and stem cells include CD3, CD8, CD 19, CD20, CD34, CD80, CD86, glycophorin A, MAP2, nestin, pankeratin, and vWF. The expression of certain markers may increase in a stem cell relative to a monocyte. Stem cell-specific markers that may increase include CD117, DPPA5, HES-1, Oct-4, SCF, and SSEA-4. The stem cell may be CD34−.

d. Identifying a De-Differentiated Cell

The de-differentiation of the monocyte into a multipotent stem cell may be identified by alternations in the rate of cell proliferation, cell cycle, or gene expression when cultured under specific conditions. After approximately 4-8 days in culture, the confluency of the LIF/M-CSF-treated cell may be greater than 75%, 80%, 85%, 90%, or 95%. After approximately 3 days in culture, the monocyte may have de-differentiated into a stem cell, as evidenced by changes in cell proliferation and gene expression. The percentage of de-differentiated cells in a population of cells may be at least 40, 50, 60, 70, 80, or 90% of the total number of cells. The population of stem cells may be maintained by continued culture in the presence of the growth factors, LIF and M-CSF.

The stem cell may be preserved indefinitely by contacting the cell with a cryopreservative agent and freezing the cell. The frozen cell may be stored at an ultra low temperature or in liquid nitrogen.

### 2. Using the Stem Cell

The stem cell may be differentiated into another cell type by the addition of the appropriate growth factors or hormones. As an example, the stem cell may be differentiated into a neuronal cell by contact with NGF, brain-derived neurotrophic factor, neurotrophin-3, basic fibroblast growth factor, pigment epithelium-derived factor, retinoic acid, and combinations thereof. The stem cell may be differentiated into an endothelial cell by contact with VEGF, IGF, BFGF, and combinations thereof. The stem cell may be differentiated into an epithelial cell by contact with EGF, BMP-4, activin, elevated calcium concentrations, retinoic acid, sodium butyrate, vitamin C, hexamethylene bis acetate, phorbol 12-myristate 13-acetate (PMA), teleocidin, interferon gamma, staurosporin, and combinations thereof. The stem cell may be differentiated into a macrophage or a T cell by contact with LPS, IL-2, IL-4, IL-12, IL-18, CD3 antibody, PMA, teleocidin, interferon gamma, and combinations thereof. The stem cell may be differentiated into a hepatocyte by contact with HGF, retinoic acid, oncostatin M, phenobarbital, dimethyl sulfoxide, dexamethasone, dibutyryl cyclic AMP, and combinations thereof.

The expression of cell lineage-specific markers may increase in a stem cell relative to a monocyte. Non-limiting examples of embryonal carcinoma (EC)-specific markers that may increase over time include Flk-1, TIE-2, and VE-cadherin. Non-limiting examples of hematopoietic-specific markers that may increase over time include TAL-1, GATA-2, and GATA-3. Non-limiting examples of cardiac-specific markers that may increase over time include NKX2.5, NKX2.2, and CD105. Non-limiting examples of pancreatic-specific markers that may increase over time include IPF1, insulin, PAX-4, IGF2, and glucagon. Non-limiting examples of endoderm-specific markers that may increase over time include AFP and ALB. Non-limiting examples of smooth muscle-specific markers may typically increase over time include SM1, SM22A, and PDGFRB.

The other cell types or differentiated cells derived from the stem cell may be used, by way of non-limiting example, to replenish or stimulate (induce) the replenishment of a cell population that has been reduced or eradicated by a disease or disorder (e.g., cancer), to treat a disease or disorder (e.g., a cancer therapy), or to replace damaged or missing cells or tissue(s). By way of example, neuronal tissue damaged during the progression of Parkinson's disease, endothelial cells damaged by surgical incisions, macrophage cells affected by Gaucher's disease, epithelial cells damaged from skin burns, hepatocytes damaged as a result of cirrhosis, pancreatic islet β-cells damaged by type I diabetes, or cardiac cells damaged by heart disease may be replenished or stimulated to replenish cells differentiated from these stem cells. Moreover, since these stem cells may be derived from the peripheral blood of the same individual who will later receive the stem cell or their derivatives, immunosuppression may not be necessary.

### 3. Compositions

Also provided herein are compositions comprising the stem cell or differentiated cells derived from the stem cell. The composition may comprise a plurality of the stem cell, which may be more than 1×10⁶ of the stem cell. The stem cell may express a marker, which may be CD117, DPPA5, HES-1, Oct-4, SSEA-4, or a combination thereof. The stem cell may have a characteristic, which may be CD11b+, CD14+, CD34−, CD45+, CD90−, CD117+, DDPA5+, HES-1+, Oct-4+, SSEA-4+, CD135−, or a combination thereof.

As various changes could be made in the above compounds, methods, and products without departing from the scope of the invention, it is intended that all matter contained in the above description and in the examples given below, shall be interpreted as illustrative and not in a limiting sense.

### EXAMPLE 1

### Generation of Monocyte-Derived Stem Cells (MDSCs)

Isolation of Monocytes. Monocytes were isolated from adult human peripheral blood using a single-step discontinuous Ficoll gradient. During this procedure, peripheral blood monocytes are localized to the interface between the blood plasma and the separation medium. To help maintain the interface, LeucoSep centrifuge tubes, which contain a positioned porous membrane barrier, were used. LeucoSep tubes (30-ml) were prepared by adding 15 ml of Lymphocyte Separation Buffer (Cat. no. 25-072-cv, Mediatech Cellgro) and centrifuging at 1000×g for 30 sec at room temperature to drive the buffer through the membrane barrier. Then 15 ml of blood and 30 ml of 1×HBSS (Hanks Balanced Salt Solution) with 2 mM EDTA were added to each tube. The tubes were centrifuged at 1000×g for 10 minutes at 4° C. After this centrifugation step, the enriched cell fraction containing lymphocytes and monocytes was located above the membrane barrier. The tubes were carefully removed from the rotor to minimize disruption of the layers. The enriched cell fraction was carefully removed with a Pasteur pipette and transferred to a 50-ml centrifugation tube and the tube filled to 50 ml with 1×HBSS that does not include Ca²⁺ and Mg²⁺ (Cat. No. 21-022-cm, Mediatech Cellgro).

The cells were centrifuged at 150×g for 15 minutes at room temperature, and the supernatant was removed. Then 10-15 ml of Red Blood Cell Lysis Buffer (Cat. No. R7757, Sigma-Aldrich) was added to the pelleted cells to remove any red blood cells that may contaminate the mononuclear cell layer. After 2 minutes, 40 ml of 1×HBSS was added to the cells, which were then spun at 150×g for 15 minutes at room temperature. The cell pellet was washed two more times with 50 ml of 1×HBSS to remove residual lysis buffer. The final pellet was resuspended in AIM V medium (Invitrogen), which is a serum-free medium that contains L-glutamine and streptomycin sulfate at 50 μg/ml. Cell density was determined using a Vi-CELL Cell Analyzer (Beckman Coulter).

De-differentiation into Monocyte-Derived Stem Cells (MDSCs). The isolated monocytes were seeded on a variety of plate formats at a density of 1-2×10⁶/ml. At this density, the cells were >75% confluent after 6 days in culture. Table 1 presents the different plates and total number of cells when plated at a density of 1×10⁶ cells/cm². The cells were plated in a 2:1 mixture of Megacell DMEM/F12 medium (Cat. No. M4192, Sigma-Aldrich) and AIM V medium and cultured overnight at 37° C. and 5% CO₂. The Megacell DMEM/F12 medium is a serum-free media based on the standard published basal formulation, but is further supplemented with buffers and sodium pyruvate. Sodium selenite, rh-Insulin, human transferrin, and fatty acids have been added to allow for serum reduction. It contains 4,500 mg/L D-glucose. Generally, it was further supplemented with 4 mM L-glutamine and penicillin-streptomyocin prior to use.

After 24 hours, the culture medium was removed and the cells were gently washed three times with 1×HBSS containing 2 mM EDTA. De-differentiation medium was added. The de-differentiation medium consisted of Megacell DMEM/F12 or LDMEM (low glucose DMEM) or HDMEM (high glucose DMEM) containing 10 ng/ml leukocyte inhibitory factor (LIF; Cat. No. LIF1010, Chemicon) and 25 ng/ml macrophage colony-stimulating factor (M-CSF; Cat. No. GF053, Chemicon). After three days, the medium was removed and replaced with fresh de-differentiation medium. After 6 days in culture the cells had de-differentiated into monocyte-derived stem cells. Cultures grown for longer than 10 days tended to develop into multinucleated osteoclastic giant cells and endothelial cells. Cells grown in the absence of LIF and M-CSF remained quiescent and did not de-differentiate.

TABLE 1
Total cells plated on the different types of plates.

| Dish type | Area | Cell Density | Total Cells | |
|---|---|---|---|---|
| 15 cm plate | 176 cm² | 1 × 10⁶ cells/cm² | 176 × 10⁶ | cells/plate |
| 10 cm plate | 78 cm² | 1 × 10⁶ cells/cm² | 78 × 10⁶ | cells/plate |
| 6 well dish | 9.5 cm² | 1 × 10⁶ cells/cm² | 9.5 × 10⁶ | cells/well |
| 24 well dish | 1.9 cm² | 1 × 10⁶ cells/cm² | 2.0 × 10⁶ | cells/well |
| 48 well dish | 1.1 cm² | 1 × 10⁶ cells/cm² | 1.1 × 10⁶ | cells/well |
| 1 well chamber | 8.6 cm² | 1 × 10⁶ cells/cm² | 8.6 × 10⁶ | cells/well |
| slide | | | | |
| 4 well chamber | 1.7 cm² | 1 × 10⁶ cells/cm² | 1.7 × 10⁶ | cells/well |
| slide | | | | |
| 8 well chamber | 0.7 cm² | 1 × 10⁶ cells/cm² | 0.7 × 10⁶ | cells/well |
| slide | | | | |

Dispersion, Freezing and Thawing of MDSCs. Adherent cells (known as MDSCs) were removed by treating the cultures with 0.5% lidocaine for 1-2 minutes. Concentrations of lidocaine greater than 1% caused an increase in cell death and a decrease in the overall cell proliferation rate. (Trypsin/EDTA and collagenase were also used to disperse the cells.) The cells were dispersed by gentle scraping and transferred to a new tube. Two volumes of Megacell DMEM/F12 or LDMEM or HDMEM were added to neutralize the lidocaine and the cells were centrifuged at 150×g for 15 minutes at room temperature. The supernatant was removed and fresh Megacell DMEM/F12 or LDMEM or HDMEM was added. To freeze the cells, 500 μl of DMSO Freezing Medium (Cat. No. 210002, Bioveris Corp.) was added to a 500 μl aliquot of 1×10⁶ cells. The tube was mixed well, frozen in an ethanol-freezing chamber, and placed at −80° C. overnight. The tube was transferred to liquid nitrogen for long-term storage. To thaw the cells, a vial of frozen cells was gently swirled in a 37° C. water bath and the cells were transferred to a 15-ml tube. Four ml of Megacell DMEM/F12 or LDMEM or HDMEM at room temperature (approximately 22° C.) was slowly added and gently mixed by swirling. The cells were spun at 150×g, the supernatant was removed, and the cells were resuspended in 2.5 ml of culture medium. The cells were ready to be plated and cultured. Cell viability was typically >90%.

### EXAMPLE 2

### Growth in Different Medium Formulations

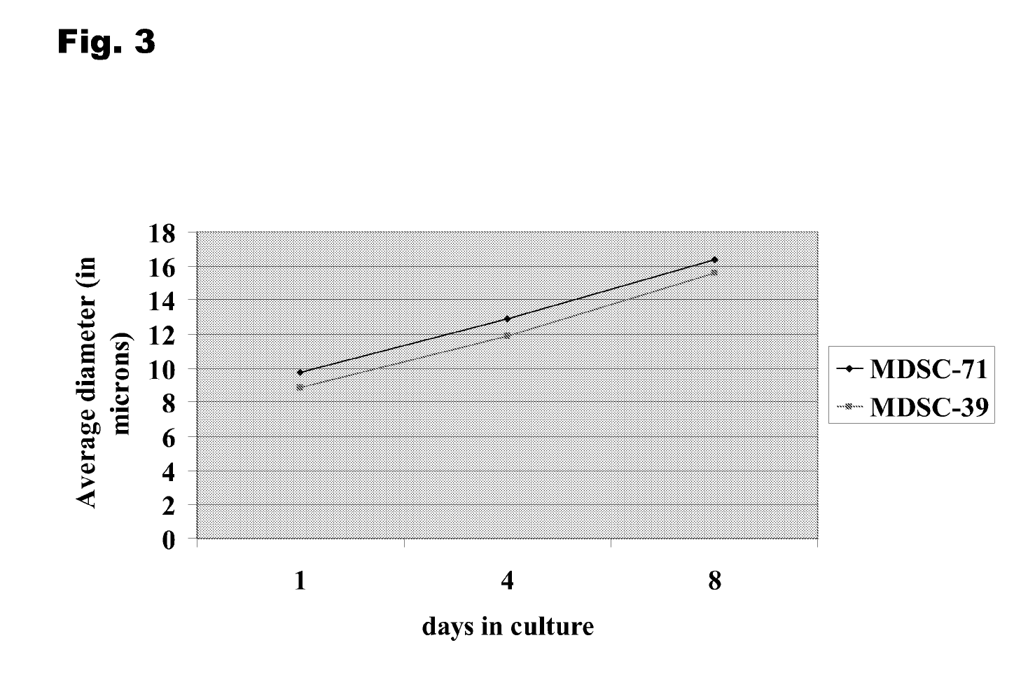

To determine the optimal conditions for growth and de-differentiation, several different medium compositions and serum levels were examined. Monocytes were derived as described in Example 1; they were plated in AIM V medium and cultured overnight at 37° C. The cells were then transferred to and grown in five different medium formulations: HDMEM, LDMEM, AIM V, RPMI, or IN VIVO 15 media. Each formulation was supplemented with 0, 5, 10, or 20% FBS and the two de-differentiation agents, 10 ng/ml LIF and 25 ng/ml M-CSF. The cells were grown for 6 days, with the medium changed at day 3. There was no difference in the percentage of MDSCs among the different conditions, but the total number of cells varied significantly among the different conditions. As shown in FIGS. 3A and 3B, growth in the presence of LDMEM or HDMEM and 10-20% FBS resulted in much higher total number of total cells per plate.

### EXAMPLE 3

### Growth on Different Substrates

To determine whether the substrate affected growth and de-differention, isolated monocytes were plated on fibronectin, gelatin, collagen, poly-lysine, or L-ornithine coated plates. The cells were grown in de-differentiation medium for 6 days, with the medium changed at day 3. Cells were collected by treatment with 0.5% lidocaine with gentle scraping and counted with a Vi-CELL cell counter. There was a small increase in the total numbers of cells grown on fibronectin or gelatin-coated plates (5-15% increase in total cell number) after 3 and 6 days in culture. The percentage of MDSCs was not significantly changed among the different treatments.

When culturing cells on different brands of polystyrene tissue dishes, it was discovered that there was a 50% increase in the initial adhesion and growth of cells on FALCON integrid vacuum gas plasma treated plates, as compared to NUNC and other brands of plates. There was also a higher percentage of MDSCs generated on the FALCON plates, e.g., 90% on FALCON plates compared to approximately 50% on NUNC plates at the same time point.

### EXAMPLE 4

### Cell Growth and Cell Size Analysis

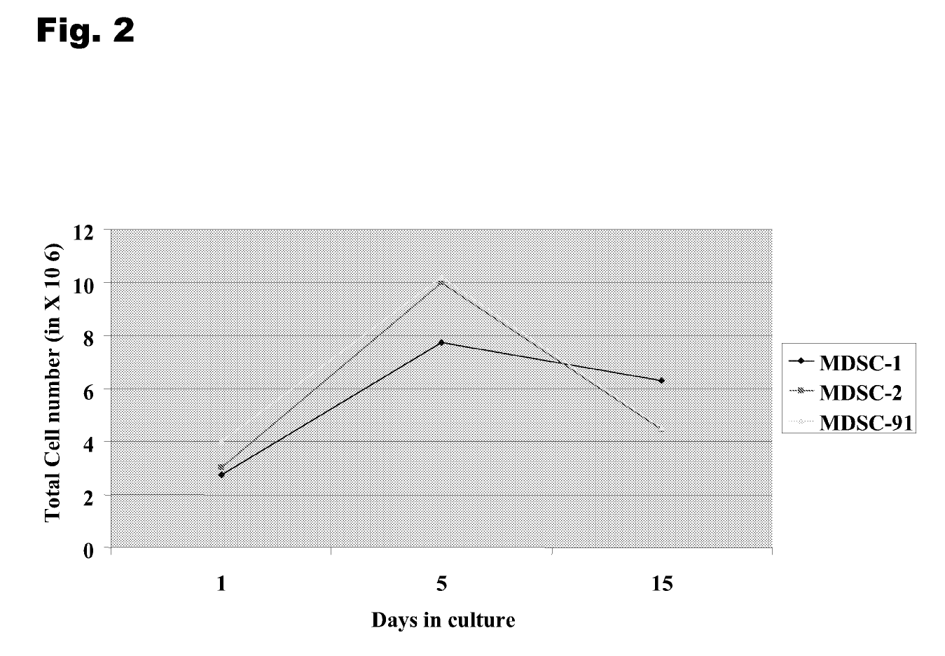

To characterize the growth and proliferation of MDSCs, the total cell numbers and average cell diameters were determined. Several different preparations of monocytes were isolated essentially as described in Example 1 and grown in the presence of de-differentiation medium for 12-15 days, with the medium changed every three days. There was an increase in total number of cells during the de-differentiation phase (day 1 to day 6), after which the cell count decreased (FIG. 2). The diameter of the cells increased from approximately 9-10 microns to approximately 16 microns during the first 8 days in culture, after which the size of the cells stabilized (FIG. 3).

### EXAMPLE 5

### Cell Cycle Analysis

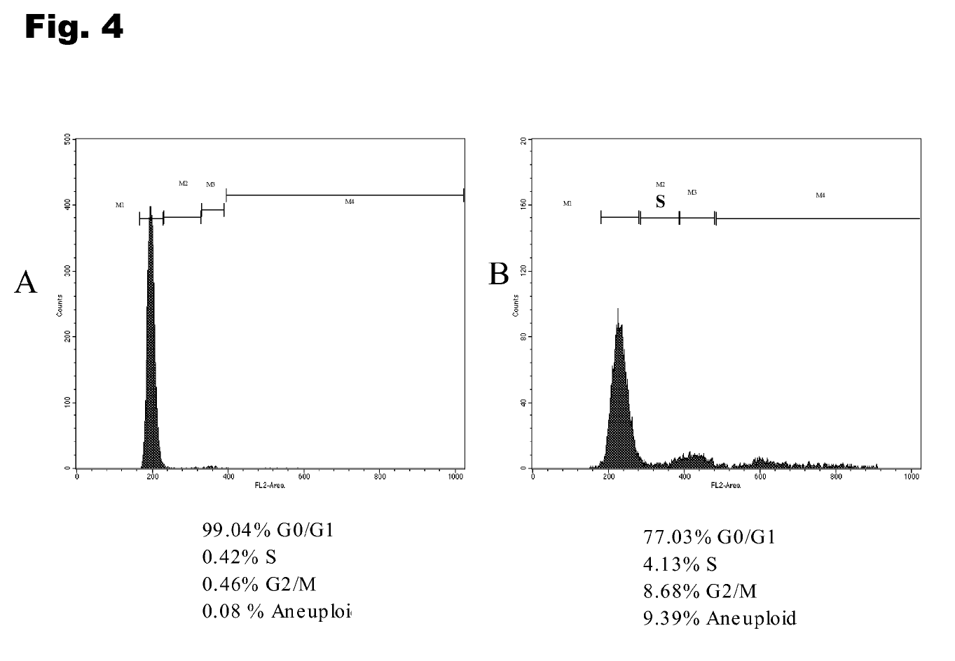

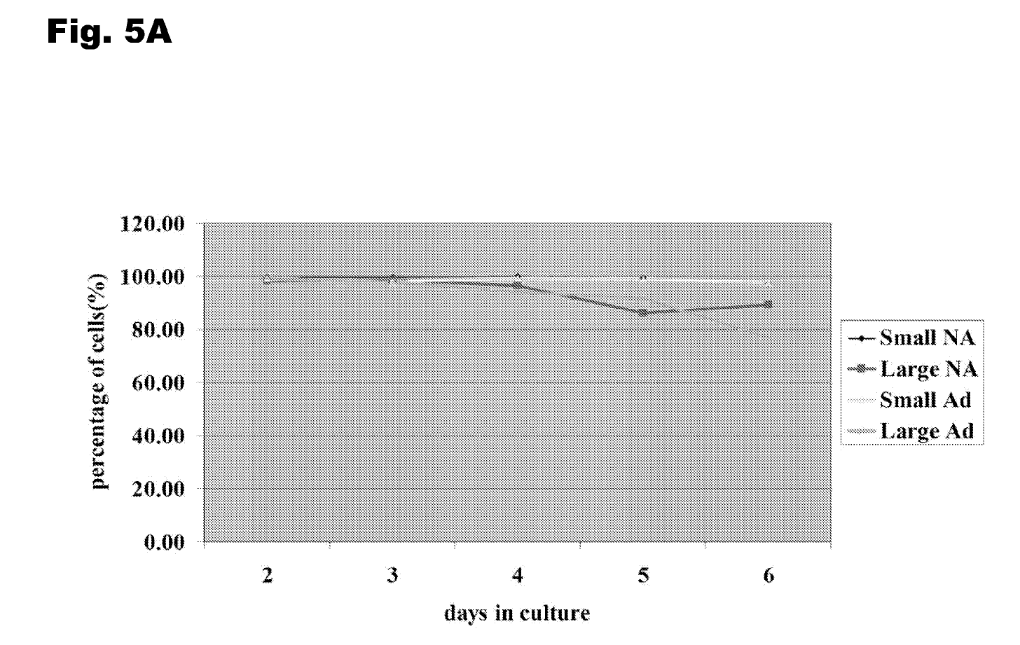

To examine changes in the cell cycle as the monocytes de-differentiated into MDSCs, flow cytometry was used. This analysis also provided the opportunity to examine the growth and de-differentiation of MDSCs during long term culturing. For these experiments, monocytes were grown in 6-well plates in the presence of de-differentiation medium for 6 days, and cells were removed from individual wells at various time points for analysis. The following cell types were characterized: small non adherent, large non adherent, small adherent, and large adherent. Panels A and B of FIG. 4 present the cell cycle analysis of large adherent cells (also known as MDSCs) on day 2 and day 6, respectively. At day 2, the cells were quiescent, with >99% in the G1/G0 phase. By day 6, a significant percentage of cells had re-entered the cell cycle, as evidenced by the increased percentages of cells in S or G2/M phases of the cell cycle. Binucleated cells were identified mainly in the G2/M phase of the cell cycle; these cells were composed of greater than 1 nuclei per cell. However, cells that contained greater than 4n of nuclei DNA were classified as aneuploid.

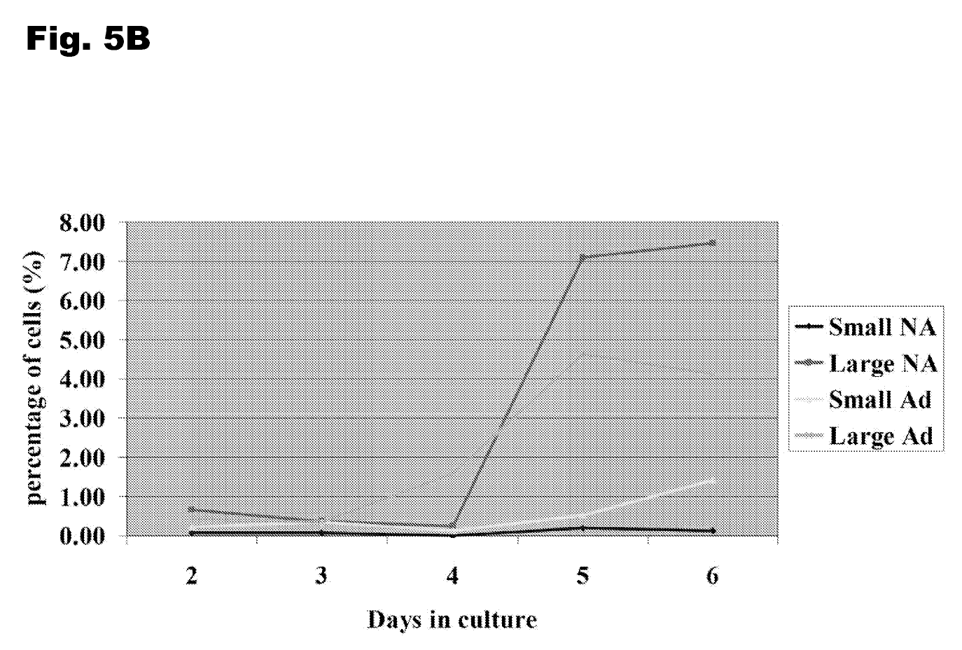

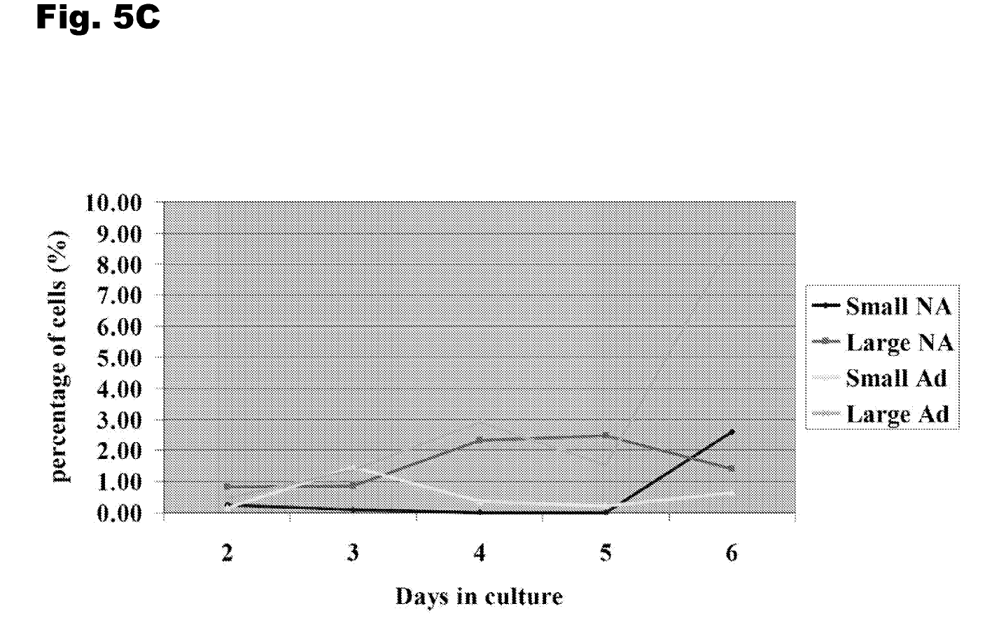

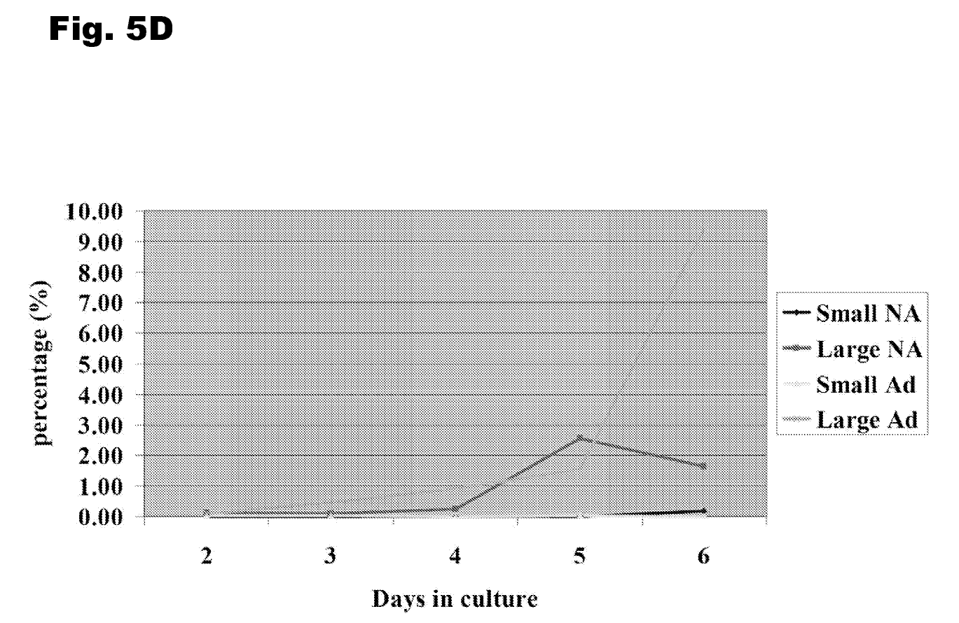

FIG. 5 shows a detailed analysis of the percent of each type of cell in the different phases of the cell cycle during days 2-6 of the de-differentiation process. By days 5-6 there is a shift in the percentage of cells in S and G2/M phases of the cycle. The percentage of aneuploid cells also increased over time. The growth analysis (see Example 4) and this cell cycle analysis suggest that the MDSCs generated by this procedure were consistent with the characteristics of a population of slowly dividing cells.

### EXAMPLE 6

### Phenotypic Analysis: Flow Cytometry

To characterize the phenotypic profiles of the MDSCs during their growth and de-differentiation, they were stained for cell lineage-specific and stem cell-specific markers. Monocytes were collected and cultured (up to 25 days) essentially as described in Example 1. At each time point, cells were collected, washed, and resuspended in Staining Buffer (1×PBS with 1% FBS and 0.1% sodium azide) at a concentration of 1×10⁷ cells/ml. Up to 1×10⁶ cells were used per staining reaction in a final volume of 100-200 μl. Some cells were only stained for extracellular antigens. For these, the antibodies were diluted in Staining Buffer at the appropriate concentration (see Table 2) and added to the above-prepared cells. The tube was gently mixed and incubated for 15 minutes at room temperature in the dark. The cells were washed in 2 ml of ice-cold Staining Buffer and centrifuged for 6 minutes at 300×g. If this was the only antibody used, the cell pellet was resuspended in 200 μl of 2% paraformaldehyde and stored at 4° C.

Other cells were stained for intracellular antigens only or a mixture of extracellular and intracellular antigens. For these, the cells were fixed and permeabilized by resuspending the washed cell pellet in 2 ml of FACSLyse (Becton Dickinson). The cells were incubated for 10 minutes at room temperature in the dark, and then washed with 2 ml of ice-cold Staining Buffer. After centrifugation at 300×g for 6 minutes, the supernatant was discarded and the pellet was resuspended in 0.5 ml of FACS Permeabilization Buffer II (Becton Dickinson). The cells were incubated for 10 minutes at room temperature in the dark, and then washed with 2 ml of ice-cold Staining Buffer. After centrifugation at 300×g for 6 minutes, the supernatant was discarded and the pellet was resuspended in 100 μl of Staining Buffer. The appropriate antibodies were added at the appropriate concentration (Table 2), mixed well, and incubated for 30 minutes at room temperature in the dark. The cells were washed with 2 ml of ice-cold Staining Buffer, spun at 300×g for 6 minutes, and the cell pellet was resuspended in 300 μl of Staining Buffer. The cells were then analyzed by flow cytometry.

TABLE 2
Directly conjugated antibodies for flow cytometry

| Antibody | Vendor | Catalog Number | Dilution |
|---|---|---|---|
| ABCG2 APC | R&D Systems | FAB995A | 1:5 |
| GlycoPhorin A PE | Becton Dickinson | 340946 | 1:10 |
| CXCR3 PE | Pharmingen | 557185 | 1:10 |
| CD3 PerCP | Becton Dickinson | 340663 | 1:10 |
| CD4 PE | Becton Dickinson | 340670 | 1:10 |
| CD8 FITC | Becton Dickinson | 340692 | 1:10 |
| CD10 FITC | Becton Dickinson | 340924 | 1:10 |
| CD11b PE | Becton Dickinson | 340712 | 1:10 |
| CD14 PerCP | Becton Dickinson | 340660 | 1:10 |
| CD14 FITC | Becton Dickinson | 347493 | 1:10 |
| CD15 FITC | Becton Dickinson | 340703 | 1:10 |
| CD19 PerCP-Cy5.5 | Becton Dickinson | 340951 | 1:10 |
| CD20 FITC | Becton Dickinson | 340673 | 1:10 |
| CD33 PerCP-Cy5.5 | Becton Dickinson | 341640 | 1:10 |
| CD34 PE | Becton Dickinson | 340669 | 1:10 |
| CD45 APC | Becton Dickinson | 340942 | 1:20 |
| CD71 FITC | Becton Dickinson | 340717 | 1:10 |
| CD80 PE | Becton Dickinson | 340294 | 1:10 |
| CD86 CyChrome | Pharmingen | 555666 | 1:10 |
| CD117 PE | Becton Dickinson | 340867 | 1:10 |
| CD133 APC | Miltenyi Biotech | 120-001-123 | 1:5 |

The cells were stained for a variety of stem cell-specific and cell lineage-specific markers. A summary of the expression profile during the de-differentiation phase (day 2-6) is presented in Table 3. A summary of the long-term patterns of expression (days 5-25) of these markers is presented in Table 4. Some monocytic and hematopoietic markers (e.g., CD11b/MAC-1, CD14, CD45) are expressed in these MDSCs from the onset and throughout the culture period. CX34 expression was not detected in either short- or long-term cultures.

TABLE 3
Short-term phenotypic expression as revealed by flow cytometry.

| | d2NA | d2Ad | d3 NA | d3Ad | d4 NA | d4 Ad | d5 NA | D5 Ad | d6 NA | d6 Ad |
|---|---|---|---|---|---|---|---|---|---|---|
| CD3 | − | − | − | − | − | − | − | − | − | − |
| CD4 | + | + | + | + | + | + | + | + | + | + |
| CD8 | − | − | − | − | − | − | − | − | − | − |
| CD10 | − | − | − | − | − | − | − | − | − | − |
| CD11b (MAC-1) | +++ | +++ | +++ | +++ | +++ | +++ | +++ | +++ | +++ | +++ |
| CD14 | +++ | +++ | +++ | +++ | +++ | +++ | +++ | +++ | +++ | +++ |
| CD15 | − | − | − | − | − | − | − | − | − | − |
| CD19 | − | − | − | − | − | − | − | − | − | − |
| CD33 | − | − | − | − | − | − | − | − | − | − |
| CD34 | − | − | − | − | − | − | − | − | − | − |
| CD45 | ++ | ++ | ++ | ++ | ++ | ++ | ++ | ++ | ++ | ++ |
| CD71 (transferrin receptor | ++ | ++ | ++ | ++ | + | + | + | + | + | + |
| CD90 (Thy1) | − | − | − | − | − | − | − | − | − | − |
| CD117 (c-kit receptor) | − | − | − | − | − | − | − | − | − | − |
| CD133 | − | − | − | − | − | − | − | − | − | − |
| ABCG2 | − | − | − | − | − | − | − | − | − | − |

TABLE 4
Long-term phenotypic expression as revealed by flow cytometry.

| Marker | d5 | d10 | d15 | d19 | d25 |
|---|---|---|---|---|---|
| CD3 | − | − | − | − | − |
| CD4 | + | + | + | + | + |
| CD8 | − | − | − | − | − |
| CD11b | +++ | +++ | +++ | +++ | +++ |
| CD14 | +++ | +++ | +++ | +++ | +++ |
| CD20 | − | − | − | − | − |
| CD33 | − | − | − | − | − |
| CD34 | − | − | − | − | − |
| CD45 | ++ | ++ | ++ | ++ | ++ |
| CD71 (Transferrin | ++ | ++ | ++ | ++ | + |
| CD80 | − | − | − | − | − |
| CD86 | − | − | − | − | − |
| CD90 (Thy1) | − | − | − | − | − |
| CD117 (c-kit R) | − | − | − | − | − |
| CD133 | − | − | − | − | − |
| ABCG2 | − | − | − | − | − |
| Glycophorin A | − | − | − | − | − |

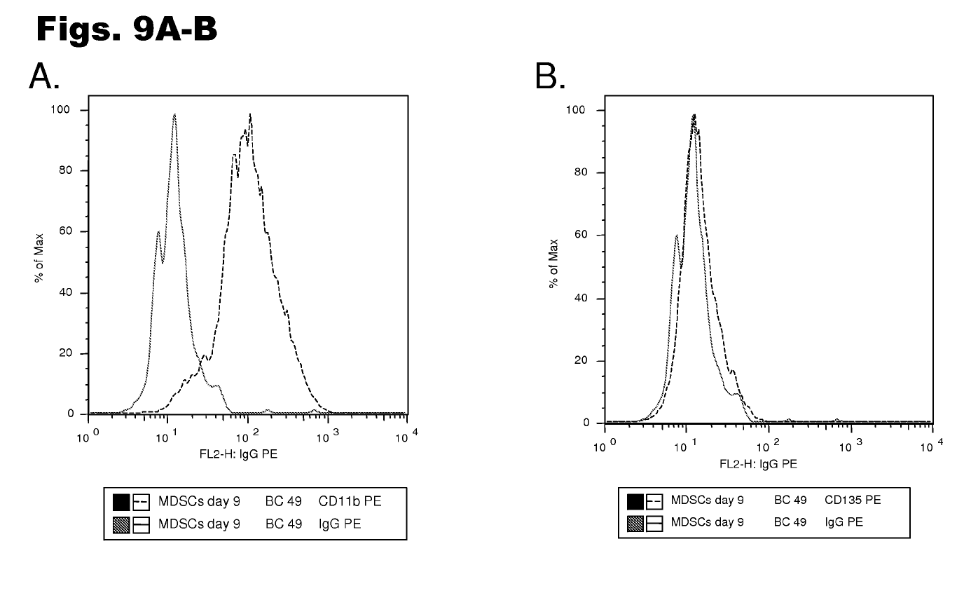

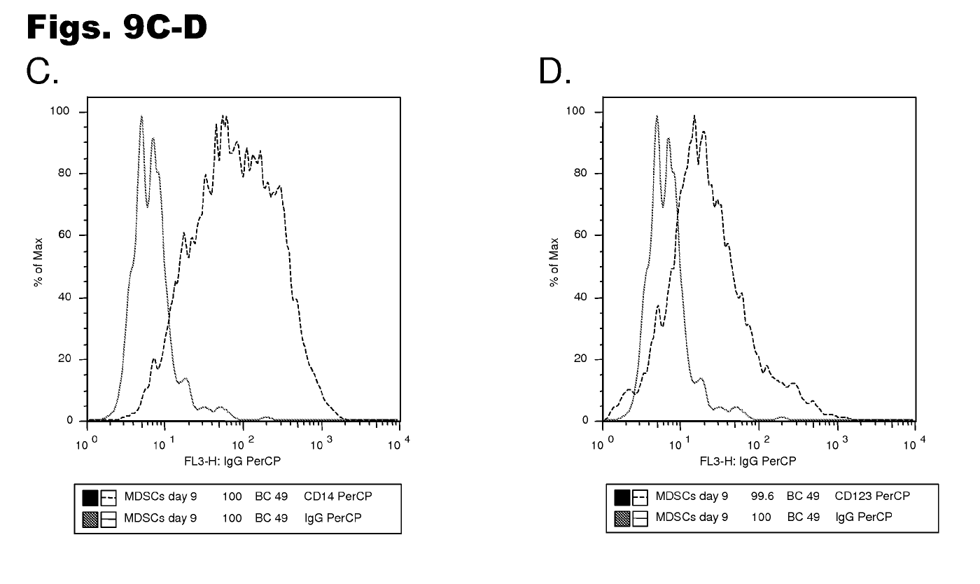

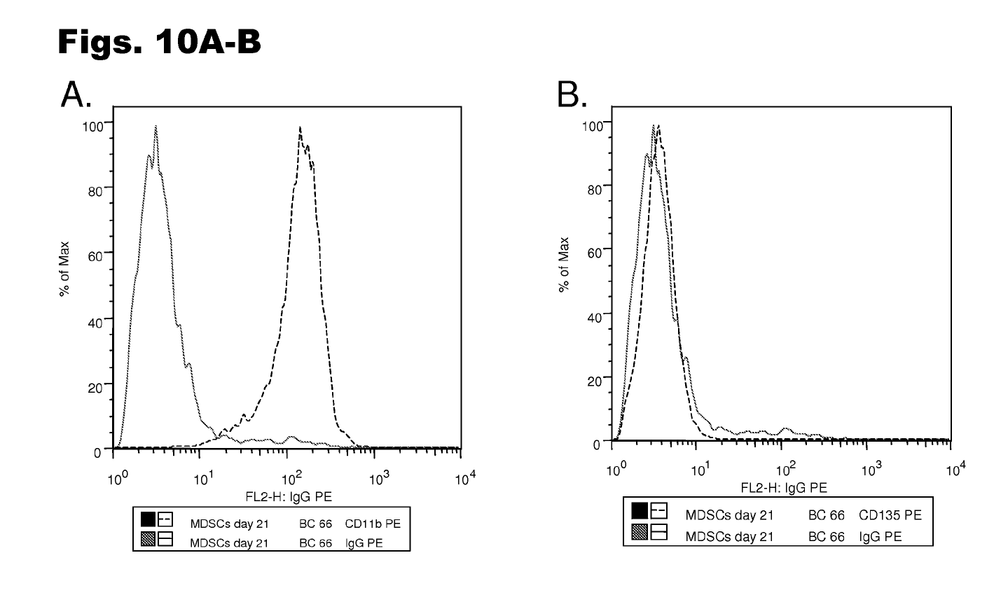

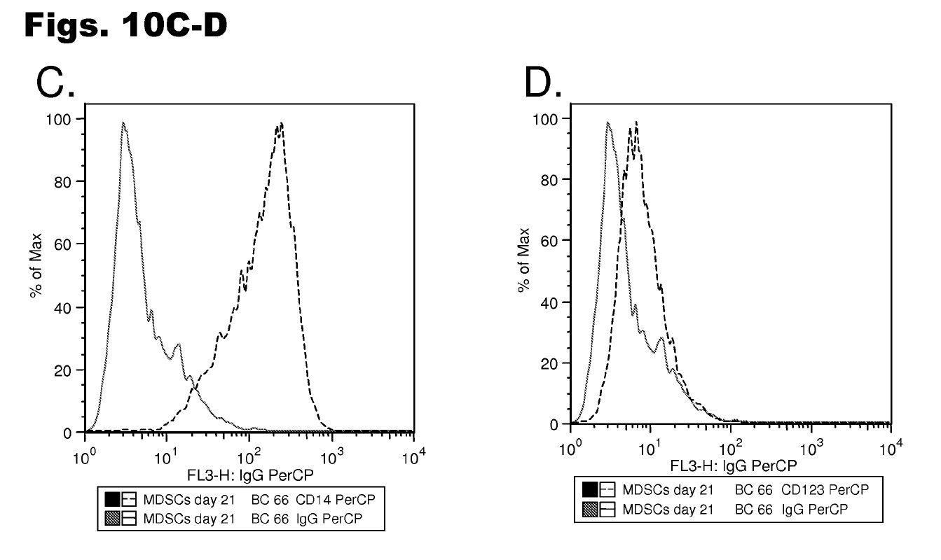

The phenotypic profile of MDSCs was further characterized during growth and differentiation by examining the expression of several other markers. MDSCs were isolated and cultured in a 6 well dish format in de-differentiation medium containing LIF and M-CSF as described above. MDSCs were then stained with antibodies against CD11b (MAC-1), CD14, CD123 (IL3R), and CD135 (Flk-2), and then analyzed by flow cytometry at day 9 (FIG. 9) and day 21 (FIG. 10).

FIG. 9 shows that MDSCs expressed high levels of CD11b (FIG. 9A) and CD14 (FIG. 9C), consistent with marker expression in a monocyte lineage. MDSCs also expressed CD123 at day 9 (FIG. 9D). MDSCs did not express CD135, suggesting a lack of Flk-2 expression (FIG. 9B).

FIG. 10 shows that, consistent with a monocyte lineage, MDSCs expressed high levels of CD11b (FIG. 10A) and CD14 (FIG. 10C) at day 21. In contrast to day 9, MDSCs expressed low levels of CD 123 at day 21 (FIG. 10D). As at day 9, MDSCs did not express CD 135 at day 21 (FIG. 10B).

In summary, high-levels of CD11b and CD14 were expressed in MDSCs at all time points measured, and CD135 (Flk-2) expression was absent in MDSCs at all time points measured. CD123 (IL3R) was positive early during de-differentiation (day 9), and lost expression intensity over time. By day 21, cultured MDSCs exhibited barely detectable levels of CD123.

### EXAMPLE 7

### Phenotypic Analysis: Immunofluorescence Staining

To better visualize the time course of activation of stem cell-specific markers, in particular, immunofluorescence staining was performed. Monocytes were isolated and cultured in 8-chamber slides using the method described in Example 1. For each time point, cells were collected, washed in Wash buffer (PBS+1% BSA) to remove any remaining medium. The cells were fixed in 200 μl of freshly made 4% formaldehyde (in PBS) for 20 minutes at room temperature, and then washed in Wash Buffer. Cells were permeabilized by adding 200 μl of FACS Permeabilization Buffer II, incubating for the appropriate time at room temperature, and washing three times in Wash Buffer. Cells were stained for specific markers by incubating with primary antibodies diluted in 200 μl of Wash Buffer (see Table 5) for 3-4 hours at room temperature, washing three times in Wash Buffer, incubating with diluted secondary antibodies for 1 hour at room temperature in the dark, and washing three times in wash buffer. Incubating in 100 μl of 10 μg/ml DAPI for 5 minutes at room temperature stained the DNA of the cells, the cells were then washed three times in wash buffer to remove any residual DAPI stain. After washing cells, an anti-fade reagent was then added to the cells to enhance fluorescent detection.

Cells stained only for extracellular markers were fixed, stained with antibodies, permeabilized for 5 min, and stained with DAPI. Cells stained only for intracellular markers or for both intra/extracellular markers were fixed, permeabilized for 30 minutes, stained with antibodies, and stained with DAPI.

Table 6 summarizes the phenotypic expression patterns during the de-differentiation phase. The expression of three stem cell-specific markers (i.e., HES-1, SSEA4, and CD117) increased over time. Initially, these markers had no or low levels of expression, but their levels increased beginning around days 4. HES-1 and SSEA4 are primitive stem cell markers, and CD117 (c-kit receptor) is normally expressed by stem cells and during embryogenesis. Table 7 presents the long-term patterns of expression of these lineage- and stem cell-specific markers. Together, these data and the flow cytometry data revealed that CD14, CD45, and CD11b expression remained constant, and CD34 was never detected in these MDSCs.

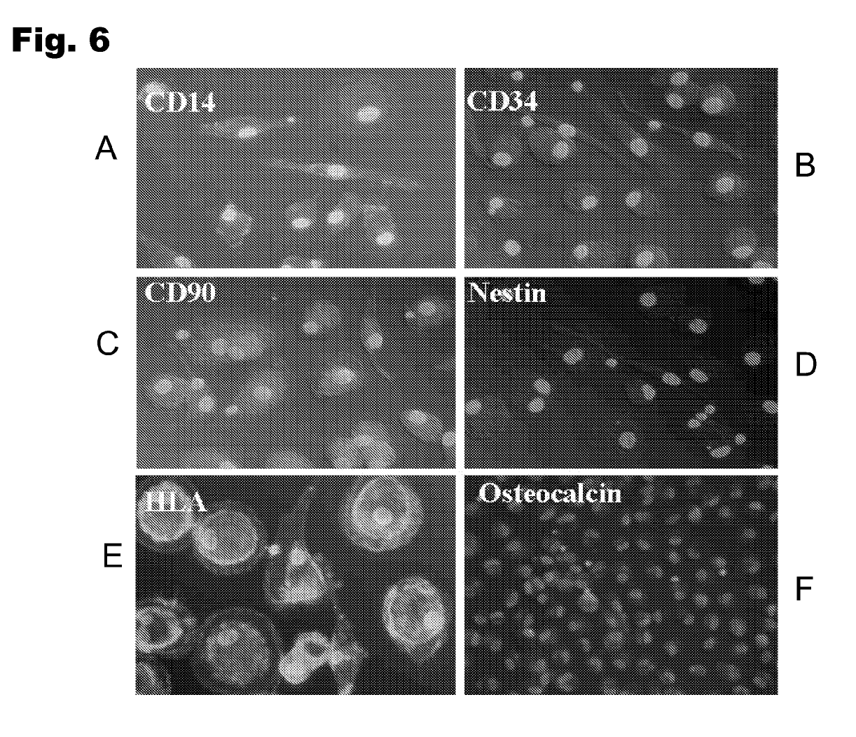

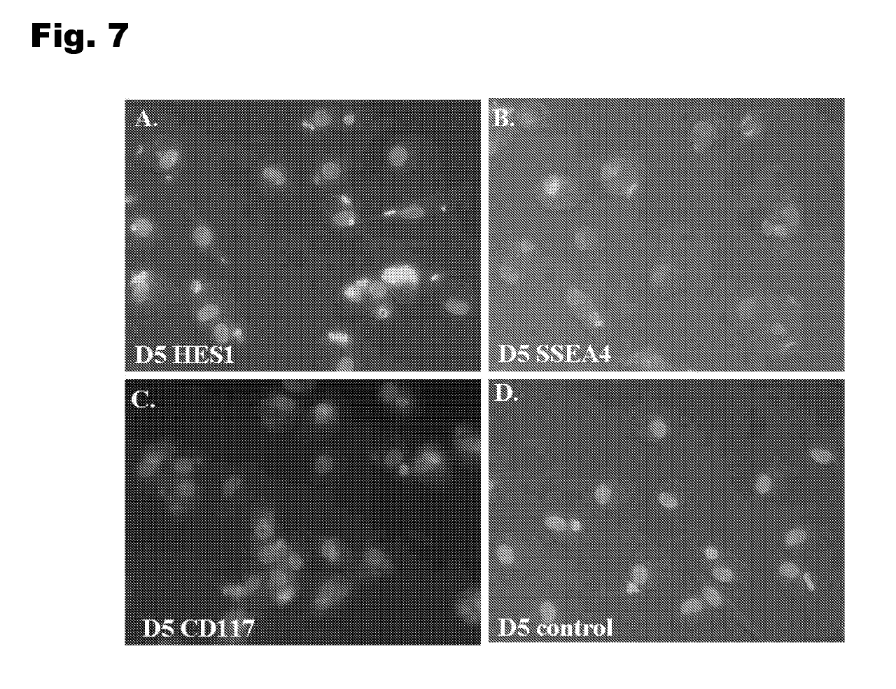

FIG. 5 presents images of cells stained for lineage-specific markers at day 9. The cells had low levels of CD14 and osteocalcin, high levels of HLA, and no CD34, CD90 and nestin expression. FIG. 6 presents images of cell stained for the stem cell-specific markers, HES-1, SSEA4 and CD1 17, at day 5.

TABLE 5
Antibodies Used For Immunofluorescence Experiments

| | | | | Catalog | |
|---|---|---|---|---|---|
| Antigen | Clone | Isotype | Vendor | Number | Concentration |
| CD3 | UCHT-1 | Mouse IgG1 | Pharmingen | 555330 | 1:100 |
| CD11b (MAC-1) | M1/70 | Rat IgG2b | Chemicon | MAB1387Z | 1:100 |
| CD14 | UCHM-1 | Mouse IgG2a | Chemicon | CBL453 | 1:100 |
| CD34 | 581 | Mouse IgG1k | Pharmingen | 555820 | 1:100 |
| CD45 | 69 | Mouse IgG1 | BD Transduction Labs | 610266 | 1:100 |
| CD90 (Thy-1) | F15-42-1 | Mouse IgG1 | Chemicon | CBL415 | 1:100 |
| CD117 (c-kit) | YB5.B8 | Mouse IgG1 | Chemicon | MAB1162 | 1:100 |
| a-fetoprotein | 2X2 | Mouse IgG2a | USBiological | F4100-04 | 1:100 |
| C-Peptide | C-PEP-01 | Mouse IgG1 | Chemicon | CBL94 | 1:100 |
| Cytokeratin-7 | OV-TL 12/30 | Mouse IgG1 | Chemicon | MAB3554 | 1:100 filter each time |
| E-cadherin | 67A4 | Mouse IgG1 | Chemicon | MAB3199 | 1:100 |
| Glut-2 | Polyclonal | Rabbit | Chemicon | AB1342 | 1:500 |
| HES-1 | Polyclonal | Rabbit | Chemicon | AB5702 | 1:200 |
| HLAabc | 22.64.4 aka PHM-4 | Mouse IgG2b | Chemicon | MAB1275 | 1:100 |
| Human Islet Cells | 3D3 | Mouse IgM | Cymbus | CBL400 | 1:100 |
| MAP-2 | | Mouse IgG1 | Chemicon | MAB378 | 1:200 |
| Nestin | 10C2 | Mouse IgG1 | Chemicon | MAB5326 | 1:200 |
| NF | Polyclonal | Rabbit | Chemicon | AB1983 | 1:100 |
| NSE | 5E2 | Mouse IgG2a | Chemicon | MAB324 | 1:100 |
| Osteocalcin | Polyclonal | Rabbit | Chemicon | AB1857 | 1:200 |
| Pankeratin | AE1/AE3 | Mouse IgG1 | Chemicon | MAB3412 | 1:100 |
| Somatostatin | Polyclonal | Rabbit | Chemicon | AB5494 | 1:100 |
| SSEA4 | MC-813-70 | Mouse IgG3 | Chemicon | MAB4304 | 1:100 |
| VEGF-R3 | Polyclonal | Rabbit | Chemicon | AB1875 | 1:100 |
| VEGF-R3 | 54703 | Mouse IgG1 | R&D Systems | MAB3491 | 1:100 |
| VEGFR (KDR) | CH-11 | Mouse IgG1 | Chemicon | MAB1667 | 1:100 |
| vWF | Polyclonal | Rabbit | Chemicon | AB7356 | 1:50 |
| vWF | 21-43 | Mouse IgG1 | Chemicon | MAB3442 | 1:100 |

| | | Catalog | |
|---|---|---|---|
| | Vendor | Number | Concentration |
| Secondary | | | |
| Antibodies | | | |
| Donkey anti-MsIgG (H + L) F(ab)2 Cy-5 | Jackson ImmunoResearch | 715-176-150 | 1:100 |
| Donkey anti-RbIgG (H + L) F(ab)2 FITC | Jackson ImmunoResearch | 711-096-152 | 1:100 |
| Donkey anti-MsIgG (H + L) F(ab)2 FITC | Jackson ImmunoResearch | 715-096-150 | 1:100 |
| Donkey anti Ms IgG Alexa488 | Molecular Probes | A21202 | 1:400 |
| Goat anti Ms IgM Alexa488 | Molecular Probes | A21042 | 1:400 |
| Goat anti-Rb IgG F(ab)2 Quantum Dot655 | Chemicon | AQ402-655 | 1:50 |
| Goat anti-Ms IgG F(ab)2 Quantum Dot525 | Chemicon | AQ400-525 | 1:50 |
| Goat anti-Rat IgG F(ab)2 Quantum Dot565 | Chemicon | AQ404-565 | 1:50 |
| CounterStain | | | |
| DAPI | Molecular Probes | D21490 | 10 ug/ml |

TABLE 6
Short-term phenotypic expression as revealed by
immunofluorescence staining.

| Scope | d2 | d3 | d4 | d5 | d6 | d8 |
|---|---|---|---|---|---|---|
| CD34 | − | − | − | − | − | − |
| CD90 | − | − | − | − | − | − |
| CD117 | − | − | + | + | + | ++ |
| CD14 | + | + | + | + | + | + |
| VEGF-R2 | − | − | − | − | − | ND |
| VEGFR3 | − | − | − | − | − | ND |
| Osteocalcin | + | + | + | ++ | ++ | +++ |
| HLAabc | +++ | +++ | +++ | +++ | +++ | +++ |
| CD11b | + | + | + | + | + | + |
| CD45 | ++ | ++ | ++ | ++ | ++ | ++ |
| HES-1 | + | + | + | ++ | ++ | ++ |
| SSEA4 | − | − | + | ++ | +++ | +++ |

TABLE 7
Long-term phenotypic expression as revealed
by immunofluorescence staining.

| Scope | d10 | d15 | d19 | d25 |
|---|---|---|---|---|
| CD34 | − | − | − | − |
| CD90 | − | − | − | − |
| CD117 | ++ | ++ | + | + |
| Nestin | − | − | − | − |
| CD14 | + | + | + | + |
| VEGF-R2 | + | + | + | + |
| VEGFR3 | ++ | ++ | ++ | ++ |
| Osteocalcin | +++ | +++ | +++ | +++ |
| NF | + | + | + | + |
| Glut-2 | − | − | − | − |
| NSE | + | + | + | + |
| MAP-2 | − | − | − | − |
| HLAabc | +++ | +++ | +++ | +++ |
| vWF | − | − | − | − |
| Pankeratin | − | − | − | − |
| CD11b | + | + | + | + |
| CD45 | ++ | ++ | ++ | ++ |

### EXAMPLE 8

### Phenotypic Analysis: PCR

To further analyze the gene expression profile of these MDSCs, RT-PCR and quantitative PCR were performed. MDSCs were cultured from 1 to 25 days in de-differentiation medium and the expression of gene products from several different cell lineages were examined. For each time point, cells were collected (1×10⁵ to 3×10⁶ cells/well) and RNA was isolated using Qiagen Rneasy Kit (Cat. No. 74103) following the manufacturer's instructions. First strand cDNA was synthesized by mixing 1 ng-5 μg of RNA with 1 μl of 500 μg/ml of oligo(dT) (Invitrogen; catalog number 55063), 1 μl of 10 mM dNTPs (Invitrogen; catalog number 18427-013), and water to equal 12 μl. The mixture was heated to 65° C. for 5 minutes and the chilled on ice. Then 4 μl of 5× First-strand buffer, 1 μl of 0.1 M DTT (Invitrogen; catalog number 18427-013), 40 units of RNaseOUT (Invitrogen; catalog number 10777-019), and 200 units of Superscript III RNaseH⁻ RT (Invitrogen; catalog number 18080-093) were added. The tube was gently mixed and incubated at 50° C. for 60 minutes. The tube was spun and the enzymes were inactivated by heating to 70° C. for 15 minutes. The concentration of cDNA was estimated using a spectrophotometer.

Primers were designed to amplify stem cell-specific, mesodermal, endothelial, neuronal, and pancreatic markers. Table 8 shows the primer sequences and sizes. Primers were designed by Primer3 software with T_{M}=60° C. PCR reactions were performed in duplicate.

TABLE 8

PCR Primer Sequences

| | | | |
|---|---|---|---|
| | | Length | SEQ ID |
| Primer Name | Sequence (5′-3′) | (bp) | NO |

| | | | |
|---|---|---|---|
| OCT4-F | GAGAACAATGAGAACCTTCAGGAG | 400 | 1 |
| OCT4-R | TTCTGGCGCCGGTTACAGAACCA | | 2 |
| CD34-f | ACCACTTCCCTCATCTCTCCTCCAA | 434 | 3 |
| CD34-R | AGGGTGAGGGAGGCAGAGACAGAAA | | 4 |
| KDR-F (VEGFR2) | TGCAGGACCAAGGAGACTATGT | 750 | 5 |
| KDR-R (VEGFR2) | TAGGATGATGACAAGAAGTAGCC | | 6 |
| TIE-2-F | ATCCCATTTGCAAAGCTTCTGGCTGGC | 400 | 7 |
| TIE-2-R | TGTGAAGCGTCTCACAGGTCCAGGATG | | 8 |
| CD31-F (PECAM1) | AGGTCAGCAGCATCGTGGTCAACAT | 800 | 9 |
| CD31-R (PECAM1) | GTGGGGTTGTCTTTGAATACCGCAG | | 10 |
| VE-CADHERIN-F | CTCTGCATCCTCACCATCACAG | 250 | 11 |
| VE-CADHERIN-R | TAGCCGTAGATGTGCAGCGTGT | | 12 |
| SM1-F | TAAACACCTGCCCATCTACTCGG | 350 | 13 |
| SM1-R | ATCTCATCATCCTGGGCTGCTGG | | 14 |
| SM22A-F | CGGCTGGTGGAGTGGATCATAG | 400 | 15 |
| SM22A-R | CCCTCTGTTGCTGCCCATCTGA | | 16 |
| PDGFRB-F | GCCTTACCACATCCGCTC | 200 | 17 |
| PDGFRB-R | TCACACTCTTCCGTCACATTGC | | 18 |
| GATA4-F | AGACATCGCACTGACTGAGAAC | 200 | 19 |
| GATA4-R | GACGGGTCACTATCTGTGCAAC | | 20 |
| NKX2.5-F | CTTCAAGCCAGAGGCCTACG | 840 | 21 |
| NKX2.5-R | CCGCCTCTGTCTTCTTCAGC | | 22 |
| AFP-F | TGCAGCCAAAGTGAAGAGGGAAGA | 200 | 23 |
| AFP-R | CATAGCGAGCAGCCCAAAGAAGAA | | 24 |
| ALB-F | TGCTTGAATGTGCTGATGACAGGG | | 25 |
| ALB-R | AAGGCAAGTCAGCAGGCATCTCATC | | 26 |
| CK18-F | GTACTGGTCTCAGCAGATTGAGGAG | 540 | 27 |
| CK18-R | GCTTCTGCTGGCTTAATGCCTCAGA | | 28 |
| CK19-F | ATGGCCGAGCAGAACCGGAA | 330 | 29 |
| CK19-R | CCATGAGCCGCTGGTACTCC | | 30 |
| GFAP-F | TCATCGCTCAGGAGGTCCTT | | 31 |
| GFAP-R | CTGTTGCCAGAGATGGAGGTT | | 32 |
| MAP2-F | GAAGACTCGCATCCGAATGG | | 33 |
| MAP2-R | CGCAGGATAGGAGGAAGAGAC | | 34 |
| MBP-F | TTAGCTGAATTCGCGTGTGG | | 35 |
| MBP-R | GAGGAAGTGAATGAGCCGGTTA | | 36 |
| GAD-F | GCGCCATATCCAACAGTGACAG | | 37 |
| GAD-R | GCCAGCAGTTGCATTGACATATA | | 38 |
| TAU-F | GTAAAAGCAAAGACGGGACTGG | | 39 |
| TAU-R | ATGATGGATGTTGCCTAATGAG | | 40 |
| TBX-5-F | GCTGGAAGGCGGATGTTTC | | 41 |
| TBX-5-R | TCGTTTTGGGATTAATGCCC | | 42 |
| SCF-F | TGGTGGCATCTGACACTAGTGA | 200 | 43 |
| SCF-R | CTTCCAGTATAAGGCTCCAAAAGC | | 44 |
| BMP-4-F | AGGAAGCAGTCTGTGTAGTGTG | 170 | 45 |
| BMP-4-R | GATGGTAGTAGAGGGATGTGGG | | 46 |
| SOX-2-F | CTTGGGCAGGCTGATAGTTTTTA | | 47 |
| SOX-2-R | TTTGTACTTGGCTCATTGCTCCT | | 48 |
| ABCG2-F | TAGTTAATCTCCTCAGACAGTAA | 161 | 49 |
| ABCG2-R | GCTACTAACCTACCTATTCATTT | | 50 |
| NESTIN-F | AGAGGGGAATTCCTGGAG | 500 | 51 |
| NESTIN-R | CTGAGGACCAGGACTCTCTA | | 52 |
| PDX-1-F | AACGCCACACAGTGCCAAAT | 142 | 53 |
| PDX-1-R | GCATGGGTCCTTGTAAAGCT | | 54 |
| DPPA5 | ATAAGCTTGATCTCGTCTTCC | 220 | 55 |
| DPPA5 | CTTGCTAGGATGTAACAAAGC | | 56 |
| ANF-F | GACAGACTGCAAGAGGCTCC | | 57 |
| ANF-R | GGAGAGGCGAGGAAGTCACC | | 58 |
| α-MYOSIN HEAVY | AAGTTCCGCAAGGTGCAG | | 59 |
| CHAIN-F | | | |
| α-MYOSIN HEAVY | TTGGCAAGCAGTGAGGTTC | | 60 |
| CHAIN-R | | | |
| MYOSIN LIGHT | CCTTCCGCATGTTTGACC | | 61 |
| CHAIN 2A-F | | | |
| MYOSIN LIGHT | GCCCCTCATTCCTCTTTCTC | | 62 |
| CHAIN 2A-R | | | |
| TROPONIN-F | CAAAGATCTGCTCCTCGCTC | | 63 |
| TROPONIN-R | AGTGGTGGCTCCCACCTAG | | 64 |
| ATP2A2-F | AAGCCAATTTTTCTGCACTG | | 65 |
| ATP2A2-R | AACAATGTTTTCTGCACAAGC | | 66 |
| BNP-F | GCCTTTTGATACTCTTACTGTGGC | | 67 |
| BNP-R | CAGGAGAAAGATTGGGAAGTGG | | 68 |
| C-KIT-F | CCAAGTCATTGTTGGATAAG | 200 | 69 |
| C-KIT-R | CTTAGATGAGTTTTCTTTCAC | | 70 |
| CD13-F | CCAGTCTAGTTCCTGATGACCC | | 71 |
| CD13-R | CAAGGCCGTTCATTGTCC | | 72 |
| CD105-F | AGTCAGCTCAGCAGCAG | | 73 |
| CD105-R | GGGGTCAACACCACAG | | 74 |
| CD133-F | ATCAGAACTGCAATCTGCACA | | 75 |
| CD133-R | AGAAGATCCCTGTCACAATTCC | | 76 |
| REX-1-F | CGCCTGTAGTCCCAGCTAC | 188 | 77 |
| REX-1-R | GATCTTGGCTCACTGCAAGC | | 78 |
| B-ACTIN-F | GCACTCTTCCAGCCTTCCTTCC | | 79 |
| B-ACTIN-R | TCACCTTCACCGTTCCAGTTTTT | | 80 |
| osteopontin-f | CTAGGCATCACCTGTGCCATACC | 600 | 81 |
| osteopontin-r | CAGTGACCAGTTCATCAGATTCATC | | 82 |
| col2a1-f | CCAGGACCAAAGGGACAGAAAG | | 83 |
| col2a1-r | TTCACCAGGTTCACCAGGATTG | | 84 |
| PPAR2-f | GCTGTTATGGGTGAAACTCTG | | 85 |
| PPAR2-r | ATAAGGTGGAGATGCAGGCTC | | 86 |
| hIns-f | GCCTTTGTGAACCAACACCTG | | 87 |
| hIns-r | GTTGCAGTAGTTCTCCAGCTG | | 88 |
| IPF1-f | CCCATGGATGAAGTCTACC | 800 | 89 |
| IPF1-r | GTCCTCCTCCTTTTTCCAC | | 90 |
| Ngn3-f | CTCGAGGGTAGAAAGGATGACGCCTC | | 91 |
| Ngn3-r | ACGCGTGAATGGGATTATGGGGTGGTG | | 92 |
| TAL-1-f | ATGGTGCAGCTGAGTCCTCC | | 93 |
| TAL-1-r | TCTCATTCTTGCTGAGCTTC | | 94 |
| GATA-2-f | AGCCGGCACCTGTTGTGCAA | | 95 |
| GATA-2-r | TGACTTCTCCTGCATGCACT | | 96 |
| Flk-1-f | ATGCACGGCATCTGGGAATC | 250 | 97 |
| Flk-1-r | GCTACTGTCCTGCAAGTTGCTGTC | | 98 |
| GATA-3-f | ACCCCACTGTGGCGGCGAGAT | | 99 |
| GATA-3-r | CACAGCACTAGAGACC | | 100 |
| AC133-f | CAGTCTGACCAGCGTGAAAA | 400 | 101 |
| AC133-r | GGCCATCCAAATCTGTCCTA | | 102 |
| INSULIN-F | GCTGGTTCAAGGGCTTTATTC | 218 | 103 |
| INSULIN-R | TGGGGCAGGTGGAGCTGGGCG | | 104 |
| GAPDH-F | AGGGGTCTACATGGCAACTG | 228 | 105 |
| GADPH-R | CGACCACTTTGTCAAGCTCA | | 106 |
| PAX-4-F | TTSCCAGGCAAAGAGGGCTGGAC | 153 | 107 |
| PAX-4R | GGCTGTGTGAGCAAGATCCTAGG | | 108 |
| IAPP-F | TAACAGTGCCCTTTTCATCTCC | 217 | 109 |
| IAPP-R(ISLET | CTGTGCCACTGAGATATAGGTCC | | 110 |
| AMYLOID | | | |
| POLYPEPTIDE | | | |
| GLUT2-F | AAACAAAGCAAATGTTCAGTGG | 176 | 111 |
| GLUT2-R | TGGGTCCCCAAAAGCTTAG | | 112 |
| NEUROGENIN-F | TCAGCAGGCAATAGATTGGG | 200 | 113 |
| NEUROGENIN-R | AAAGGAAAGGCCGTCTAGGG | | 114 |
| CARBOXYPEPTIDASE- | GATCTACCTAGTTTAATAGACCC | 148 | 115 |
| F | | | |
| CARBOXYPEPTIDASE- | TGTACTAGTTGAGAAAGCTGAT | | 116 |
| R | | | |
| IGF2-F | AGTGAGCAAAACTGCCGC | 214 | 117 |
| IGF2-R | GAAGATGCTGCTGTGCTTCC | | 118 |
| GLUCAGON-F | CTTCACAACATCACCTGCTAGC | 246 | 119 |
| GLUCAGON-R | ACAGGTTGGGGTACTTCATCC | | 120 |
| ISLET-1-F | TGAAATCCTGGGTCTCTTGG | 330 | 121 |
| ISLET-1-R | GCAATGCAAGAGCAAACAAA | | 122 |
| PANCREATIC | GACTTTCCAGCAGTCCCATA | | 123 |
| AMYLASE-F | | | |
| PANCREATIC | GTTTACTTCCTGCAGGGAAC | | 124 |
| AMYLASE-R | | | |
| GATA-4(N)-F | CTACAGGGGCACTTAACCCA | 157 | 125 |
| GATA-4(N)-R | AGAGCTGAATCGCTCAGAGC | | 126 |
| HLA-A-F | ACTCTGGAAGGTTCTCATGTG | 193 | 127 |
| HLA-A-R | AGGTGTCTCCATCTCTGTCTC | | 128 |
| KERATIN-F | CTTTTCGCGCGCCCAGCATT | | 129 |
| KERATIN-R | GATCTTCCTGTCCCTCGAG | | 130 |
| E-CADHERIN-F | AGAACAGCACGTACACAGCC | | 131 |
| ECADHERIN-R | CCTCCGAAGAAACAGCAAGA | | 132 |
| CD90(THY1)-F | AGAAGGTGACCAGCCTAACGG | 324 | 133 |
| CD90(THY1)-R | TCTGAGCACTGTGACGTTCTG | | 134 |
| CD9-F | GCTCTGGACAAACCCTGCA | 250 | 135 |
| CD9-R | AGTGGGAGTCCAAGACTCAG | | 136 |
| CD45-F | ATTTATTTTGTCCTTCTCCCA | 260 | 137 |
| CD45-R | GTTAACAACTTTTGTGTGCCAAC | | 138 |
| GLP-1R-F | TGAACCTGTTTGCATCCTTCA | | 139 |
| GLP-1R-R | ACTTGGCAAGCCTGCATTTGA | | 140 |
| CD10-F | TCAGTTTATCCTGCCCACTGATT | 350 | 141 |
| CD10-R | GGGAGCTGATGAAACTCACAAAT | | 142 |
| CD11B-F | ACAGAGCTGCCTCTCGGTGGCCA | 490 | 143 |
| CD11B-R | TTCCCTTCTGCCGGAGAGGCTACGC | | 144 |
| CD33-F | TAGCCCAGTCATTCCTAAACCAG | 296 | 145 |
| CD33-R | CTGTCCTAAGAGGCAAGAAACCA | | 146 |
| CD14-F | AGGACTTGCACTTTCCAGCTTG | 566 | 147 |
| CD14-R | TCCCGTCCAGTGTCAGGTTATC | | 148 |
| CD38-F | TTTTTAATGAGGTGGCTTTCTAACA | 241 | 149 |
| CD38-R | AGCAATCCGAGGAAACGAG | | 150 |
| CD4-F | TCAGGGAAAGAAAGTGGTGC | 138 | 151 |
| CD4-R | AAGAAGGAGCCCTGATTTCC | | 152 |
| TROPONIN1-F | TGATGTAGACGCTGCTGGTC | 136 | 153 |
| TROPONIN1-R | GGCTCCAGCACCATGATACT | | 154 |
| NSE-F | CTGCTGATCCTTCCCGATAC | 700 | 155 |
| NSE-R | ATTGGCTGTGAACTTGGACC | | 156 |
| CXCR3-F | CCACTGCCAATACAACTTCC | 401 | 157 |
| CXCR3-R | GCAAGAGCAGCATCCACATC | | 158 |
| CXCR4-F | CATCTACACAGTCAACCTCTA | 807 | 159 |
| CXCR4-R | CTAAAGAAACACAAGACAAAA | | 160 |
| CDX-2F | AGACCAACAACCCAAACAGC | 151 | 161 |
| CDX-2R | GTCACCAGAGCTTCTCTGGG | | 162 |
| HNF-3B-F | AATCATTGCCATCGTGTG | 262 | 163 |
| HNF-3B-R | CGCGGCTTAAAATCTGGTAT | | 164 |
| NKX-2.2F | TGGACGCTGTGCAGAGCCTG | NA | 165 |
| NKX-2.2R | CAGGTCCTGGGCTTTGAGCG | | 166 |
| PAX6-F | AACTGGAACTGACACACCAGG | 191 | 167 |
| PAX6-R | CCTATGCAACCCCCAGTCC | | 168 |
| OSTEOCALCIN-F | CAGTTCTGCTCCTCTCCAGG | 185 | 169 |
| OSTEOCALCIN-R | CCATCCTCCTGACACCTCC | | 170 |
| GENESIS-F | GCATCTGCGAGTTCATCAGCAAC | 157 | 171 |
| GENESIS-R | GGGTCCAGGGTCCAGTAGTTGC | | 172 |
| CD34-2F | CCTGCTCTCTTGTAATGATATAGCC | 227 | 173 |
| CD34-2R | GAGACTAGAACTGAGCTGTTTGTCC | | 174 |

For RT-PCR, 30-300 ng of cDNA was mixed with PHUSION HF buffer, PHUSION dNTPs, MgCl₂, 200 nM of each primer, and PHUSION DNA polymerase (Finnzymes). The cycling parameters were 98° C. for 30 sec, followed by 40 cycles of 98° C. for 10 sec, 58-72° C. for 10 sec, 72° C. for 20 sec 2, and a final extension at 72° C. for 5 minutes. The products were resolved in 1-3% agarose gels.

For real time (quantitative) PCR, 100 ng of cDNA was mixed with 200 nM of each primer, and 0.5 volume of SYBR green qPCR SuperMix-UDG with ROX (Invitrogen; catalog number 11744). The cycling parameters were 50° C. for 2 minutes, 95° C. for minutes, followed by 40 cycles of 60° C. for 30 seconds and 95° C. for 30 seconds. To determine the relative gene expression, the ΔCT values for controls (GADPH and β-actin) were compared to pancreatic gene expression. To calculate the percent of relative expression the following formula was used:

R.E. (relative expression)=2^{n-(ΔCT gene-ΔCT GAPDH)×100}

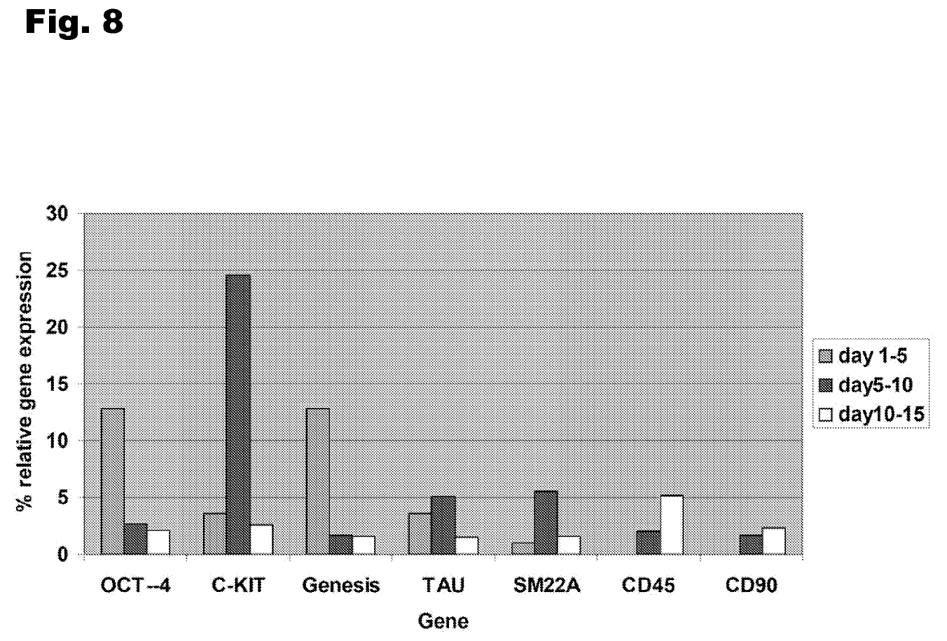

Tables 9-16 and FIG. 8 present the results of the PCR analyses. Expression of the stem cell-specific markers, OCT-4, CD117, DPPA5, SCF, and Genesis, was increased in the de-differentiated stem cells relative to the undifferentiated monocytes.

TABLE 9
Expression of Stem Cell Markers

Days in Culture

| Marker | 1 | 5 | 10 | 19 |
|---|---|---|---|---|
| OCT-4 | − | − | + | + |
| CD117 | − | + | + | + |
| DPPA5 | − | + | + | + |
| SCF | − | − | + | + |

TABLE 10
Expression of Embryonal Carcinoma (EC) Markers

Days in Culture

| Marker | 1 | 5 | 10 | 19 |
|---|---|---|---|---|
| Flk-1 | − | − | + | + |
| TIE-2 | − | + | + | + |
| CD31 | + | + | + | + |
| VE-cadherin | − | − | − | + |

TABLE 11
Expression of Hematopoietic Markers

Days in Culture

| Marker | 1 | 5 | 10 | 19 |
|---|---|---|---|---|
| CD34 | + | + | + | + |
| TAL-1 | − | − | − | + |
| GATA-2 | − | − | − | + |
| CD133 | + | + | + | + |
| GATA-3 | − | − | − | + |
| AC133 | + | + | + | + |

TABLE 12
Expression of Cardiac Markers

Days in Culture

| Marker | 1 | 5 | 10 | 19 |
|---|---|---|---|---|
| GATA4 | + | + | + | + |
| NKX2.5 | − | + | + | + |
| NKX2.2 | − | − | + | + |
| ANF | + | + | + | + |
| BNP | + | + | + | + |
| CD105 | − | − | + | + |
| TBX-5 | − | + | + | + |
| BMP-4 | + | + | + | + |

TABLE 13
Expression of Pancreatic Markers

Days in Culture

| Marker | 1 | 5 | 10 | 19 |
|---|---|---|---|---|
| IPF1 | − | + | + | + |
| PDX-1 | + | + | + | + |
| Insulin | nt | − | + | + |
| PAX-4 | − | − | − | + |
| IAPP | + | + | + | + |
| GLUT2 | + | + | + | + |
| Neurogenin | nt | nt | + | + |
| Carboxypeptidase | nt | nt | nt | + |
| IGF2 | − | + | + | + |
| Glucagon | − | − | + | + |
| Islet-1 | + | + | + | + |

TABLE 14
Expression of Endodermal Markers

Days in Culture

| Marker | 1 | 5 | 10 | 19 |
|---|---|---|---|---|
| AFP | − | + | + | + |
| ALB | − | − | − | + |
| CK19 | + | + | + | + |
| CK18 | + | + | + | + |

TABLE 15
Expression of Smooth Muscle Markers

Days in Culture

| Marker | 1 | 5 | 10 | 19 |
|---|---|---|---|---|
| SM1 | − | − | − | + |
| SM22A | − | − | − | + |
| PDGFRB | − | + | − | + |

TABLE 16
Expression of Cell Surface Markers

Days in Culture

| Marker | 1 | 5 | 10 | 19 |
|---|---|---|---|---|
| CD4 | + | + | + | + |
| CD9 | + | + | + | + |

What is claimed is:

1. A method for generating a stem cell, the method comprising:
(a) providing an isolated monocyte; and
(b) contacting the monocyte with a de-differentiation agent.

2. The method of claim 1, wherein the de-differentiation agent comprises leukocyte inhibitory factor (LIF) or macrophage colony-stimulating factor (M-CSF).

3. The method of claim 1, wherein the stem cell expresses a marker selected from the group consisting of CD117, DPPA5, HES-1, Oct-4, and SSEA4.

4. The method of claim 1, wherein the monocyte is isolated from mammalian peripheral blood.

5. The method of claim 4, wherein the mammal is a human.

6. The method of claim 5, wherein the human is an adult.

7. The method of claim 1, wherein the monocyte does not express a marker selected from the group consisting of CD117, DPPA5, Oct-4, SSEA-4, CD135, and combinations thereof.

8. The method of claim 1, wherein the stem cell expresses a marker selected from the group consisting of CD117, DPPA5, HES-1, Oct-4, SSEA-4, and combinations thereof.

9. The method of claim 1, wherein the stem cell has a characteristic selected from the group consisting of CD11b+, CD 14+, CD34−, CD45+, CD90−, CD117+, DPPA5+, HES-1+, Oct-4+, SSEA-4+, CD135−, and combinations thereof.

10. The method of claim 1, wherein the monocyte and the stem cell are grown in a serum-free medium.

11. The method of claim 1, wherein the stem cell is generated after 4-8 days in culture.

12. The method of claim 1, wherein the stem cell is contacted with a cryopreservative agent and deep-frozen.

13. An isolated stem cell, wherein the cell expresses a marker selected from the group consisting of CD117, DPPA5, HES-1, Oct-4, SSEA-4, and combinations thereof.

14. The stem cell of claim 13, wherein the stem cell has a characteristic selected from the group consisting of CD11b+, CD14+, CD34−, CD45+, CD90−, CD117+, DPPA5+, HES-1+, Oct-4+, SSEA-4+, CD135−, and combinations thereof.

15. The stem cell of claim 13, wherein the stem cell is derived from an isolated monocyte.

16. The stem cell of claim 15, wherein the monocyte is derived from mammalian peripheral blood.

17. The stem cell of claim 16, wherein the mammal is a human.

18. The stem cell of claim 17, wherein the human is an adult.

19. The stem cell of claim 13, wherein the stem cell is contacted with a cryopreservative agent and deep-frozen.

20. A composition comprising more than 1×10⁶ of the stem cell of claim 13.
